(19) United States
(12) Patent Application Publication
(10) Pub. No.: US 2002/0187130 A1
(43) Pub. Date: Dec. 12, 2002

(54) Adjuvant immune therapy in the treatment of solid tumors through modulation of signaling pathways following engagement of humoral and cell mediated responses

(76) Inventors: George Kindness, Middletown, OH (US); Brooke Schumm III, Ellicott City, MD (US); F. Timothy Guilford, Palo Alto, CA (US)

Correspondence Address: BROOKE SCHUMM, III, DANEKER, MCINTIRE, SCHUMM, PRINCE, GOLDSTEIN, ET A, 210 N CHARLES ST, SUITE 800, BALTIMORE, MD, 21201, US

(21) Appl. No.: 09/880,745

(22) Filed: Jun. 13, 2001

Related U.S. Application Data
(63) Continuation-in-part of application No. 60/263,486, filed on Jan. 23, 2001.

(51) Int. Cl. A61K 45/00; A61K 38/16

(52) U.S. Cl. 424/93.7; 514/8

(57) ABSTRACT

The invention combines a novel combination with two especially important aspects: first, the invention proposes to simultaneously stimulate response in white blood cells and a patient's tumor cells with a mitogen-challenging compound, preferably a lectin, in the preferred mode the selected lectin being phytohemagglutin (“PHA”), and second, to generate heat shock protein. A method of treatment is set out. The method of manufacturing proposed utilizes a system calculated to better insure sterility and streamline production of the cytokine modulator. A method of testing in conjunction with the therapy is also claimed utilizing clinical assessment of disease activity, patient performance status, and quality of life questionnaire. Should efficacy of a treatment fall off, particularly because of mutation or adaption, the composition and method may be re- applied. The invention is not limited to humans, but is also applicable to mammals. The composition is usable as a stand-alone composition, but preferably is used in conjunction with standard therapy such as radiation, chemotherapy or surgery, particularly surgical therapy, and in conjunction with the administration of cystine, as later defined, to enhance immune system competency.

This is a continuation in part pursuant to 37 C.F.R. 1.78 of Prov. Appl. No. 60/263,486 filed on Jan. 23, 2001 and of a provisional application bearing the above name being filed simultaneously to this specification in June, 2001.

The inventors propose a composition with immunogenic properties acting like an anti-cancer vaccine, method of treatment, and method of administration. The composition is referred to as a cytokine modulator. The composition is usable as a stand-alone composition, but preferably is used in conjunction with standard therapy such as radiation, chemotherapy or surgery, particularly surgical therapy, and in conjunction with the administration of cystine, as later defined, to enhance immune system competency. The invention combines a novel combination with two especially important aspects: first, the invention proposes to simultaneously stimulate response in white blood cells and a patient's tumor cells with a mitogen-challenging compound, preferably a lectin, in the preferred mode the selected lectin being phytohemagglutin (“PHA”), and second, to generate heat shock protein. A method of treatment is set out. The method of manufacturing proposed utilizes a system calculated to better insure sterility and streamline production of the cytokine modulator. A method of testing in conjunction with the therapy is also claimed utilizing clinical assessment of disease activity, patient performance status, and quality of life questionnaire. Should efficacy of a treatment fall off, particularly because of mutation or adaption, the composition and method may be re-applied. The invention is not limited to humans, but is also applicable to mammals. All references to humans can be applied to mammals generally and the use of you, the body or human references are not intended to be limiting.

### BACKGROUND

Current oncological research provides no clearly adequate answer for solid tumors refractory to standard therapy. Multiple therapeutic measures have been studied, but the effectiveness of any single approach to date has been limited by the heterogeneous nature of cancer and the characteristics of tumor growth.

In the formation of solid tumor mass, a single cancer cell will grow, if unchecked, to a number of cells lethal to the host. Before diagnosis can occur, occult micro-metastases already have been established at distant sites, diminishing the possible success of local treatment (surgery or radiotherapy). Efforts to reduce residual tumor burden after surgery or radiotherapy have produced mixed results, in part due to the generally inaccurate (and low) estimates of mass determined from methods currently available. Pharmacological approaches to reduction of tumor burden have proven less than satisfactory. Direct exposure of tumor in vitro to pharmacologically active anti-cancer drugs is highly predictive of in vivo response, making the process invaluable in new drug screening. To date, however, the approach has not gained widespread clinical use in guiding patient therapy.

The exponential growth pattern of tumors further decreases the ability of anti-cancer drugs to relieve tumor burden. Drug resistant tumors arise spontaneously but with a definite frequency. The number of cancer cells resistant to anticancer drugs is small initially, but resistance increases in a stepwise fashion following Gompertzian growth kinetics. A 95% probability exists of finding a drug resistant cell in the first million tumor cells, generally the volume necessary for detection with current diagnostic methods. The proportion of resistant cells increases with the growth of the tumor, progressively decreasing the likelihood of a successful response to chemotherapy drugs.

The strategy for the cytokine modulator is the deployment of the immune system to facilitate the destruction of a malignancy. This approach uses the host's immune system, a system that evolved to detect exquisitely subtle changes in molecular architecture to tell non-self from the body in order to fight cancer. The fact that many aspects of tumor microphysiology can influence the effectiveness of cancer therapy is established. The key to making immunotherapy successful as an adjuvant to standard therapy lies in helping the immune system recognize malignant cells as defective tissue.

The promise of vaccines to treat cancer dates from the early stages of the science of immunology. For the past thirty years, physicians have vaccinated thousands of cancer patients with malignant cells—either the patients' own cells or those isolated from another patient—usually irradiated preventing further growth. “Immunotherapy for Cancer.” Scientific American (September 1996). Unfortunately, most efforts offered no way to monitor the vaccine's effect on the immune system. Without such knowledge, investigators had no hope of understanding why some treatments worked while others did not. Today science offers the tools to measure such parameters including modulators of signaling pathways following engagement of humoral and cell mediated responses.

As is the case with monoclonal antibody therapies, there are now more vaccine-based therapies than anyone can test in patients. The whole-cancer-cell vaccine, whether genetically engineered or not, will give way, as outlined in this project, to cytokine modulators containing defined tumor antigens. The steady progress of the past thirty years places cytokine modulator treatment on a firm scientific basis.

Cytokines have a central role in positive and negative regulation of immune reactions. Thus the ability to predict specific immune response through cytokine stimulation and release becomes a major determinant of the type of response and eventual predictive outcome to the success or failure of a particular therapy. In addition, the pretesting(baseline) affords the unique opportunity to direct specific immune responses through the T-helper (Th) cells. Through cytokine profiling, distinct patterns of effector function can be elicited. In essence, Th1 directed cells promote macrophage (mφs) activation while Th2 cells favor antibody production. These unique patterns of effector function from cytokine profiling would help top eliminate activation of an inappropriate effector function which could lead to failure with resulting chronic immunopathology. Through customizing experimental systems the therapy can then be tailored to the individual needs of the respective patient and disease state encountered. A summary of the functions and integration of important cytokines and markers of immune function is set out in Table 1. As referenced in the preferred method of invention, changes in cytokines and markers of immune function will be important to determining efficacy of therapy.

The use of interleukin-2 [IL-2] in small amounts has been particularly successful in stimulating immune function when treatment includes the ablation of suppressor lymphocyte populations that could block responses. Mertlesman, R H, and Welte, K, “Human interleukin-2: molecular biology, physiology, and clinical possibilities,” Immunobiology (1986) 172: 400-419. In the treatment of solid tumor, the outpatient administration of very low dose IL-2 has been effective with minimal adverse effect. Alonso treated thirty patients suffering from a variety of metastatic solid tumors refractory to usual therapies with very low dose IL-2 intradermally. Alonso, K, “Interleukin-2 with immunomodulation in the treatment of human malignancy: a pilot study,” So. Med. J. (1988) 81 (Suppl. 4): 43. Results included three complete remissions and twenty partial or minor responses. With Medenica, Alonso treated thirty-eight additional patients with metastatic solid tumors refractory to usual therapies and demonstrated three complete remissions and twelve partial remissions. Medenica, R, and Alonso, K “Relationship of LAK cells and interleukin-2 in autologous tumor culture and in vivo,” J. Clin. Oncol. (1989) 8 (Suppl.): 179. Patients who demonstrated immune system exhaustion prior to therapy did not respond, hence the importance of establishing baseline cytokine levels as a predictor of immune system exhaustion.

A normally functioning immune system depends on self-recognition. T-cell immune responses generated by antigen presentation with concomitant recognition of major histocompatibility complex [MHC] gene products by T-lymphocytes in the presence of cytokine [such as IL-2], activates natural killer (NK) cell expression against cells not expressing MHC class 1.

MHC are transport molecules that carry protein linked immunologic markers from the interior of cells to the cell surface, where they are presented to the body's defense mechanisms. The immunologic markers are called antigens. The antigens are used by the T cells to distinguish cells which are part of the body from those which are not and is known as distinguishing self from non-self. The process of presentation is constant in every cell in the body and is used not only by normal cells but also by undesirable cells which by such presentation “inadvertently” present protein markers associated with disease processes, which upon recognition by cytotoxic T-cells, initiate binding which leads to identification of the cell as abnormal, and initiation of cytotoxic T-cell mediated activity.

MHC proteins are divided into two general categories with MHC class I proteins present on every nucleated cell surface in the body. The general result of the MHC class I antigens is that if a cytotoxic T cell can bind with a peptide in a MHC-peptide complex on the cell surface, that cell is producing a peptide that is not native to the host and triggers NK cell activity to remove the cell.

In this invention, the PHA stimulates the activity of white blood cells to undertake surveillance for abnormal cells. The macerated cancer cells are debris that are digested by scavenger cells such as antigen presenting cells, primarily macrophages. Those antigen presenting cells present an MHC class II marker to the macerated cancer cells. MHC class II markers are present exclusively on antigen presenting cells which are primarily B lymphocytes, dendritic cells, macrophages, and other cells that present antigens to T-helper cells. The T-helper cells in turn release cytokines which in turn regulate other components of the immune response. This would include antibody production and NK cell activity. The general result of MHC class II markers that emerge on antigen presenting cells is that the markers initiate a sequence of events, including cytokine directed signaling to immune-function cells, which are then directed to undesirable cells with the result that antibodies and targeted immune responses are directed to previously identified abnormal cells, in this case cells like the macerated cancer cells. Heat shock proteins, more accurately shock proteins (in this invention, generated by heat), generate a synergism to act with the cytokines to direct immune system targeting against the alien cancer cells against which immune response is being mounted. Overall, the number of NK cells and T-helper cells is upregulated amidst the general immune system response.

It is clear that cytokines are necessary for full lymphocyte activation. In particular, full and sustained activation of naive T cells depends upon the previously mentioned synergism of co-stimulatory signals transduced through molecules such as CD28. These second signals activate distinct signal transduction pathways, which are integrated with T cell receptor (TCR)—signals to generate effector T cells, and so drive distinct patterns of effector function. This generates cumulative effects against the aberrant cells.

Previous work suggests that cytotoxic cells effective against cancer cells will have a Th-1 profile of cytokine secretion. Alonso, K., Page, R., Kindness, G., McLaren, J. “Concomitant use of autologous tumor vaccine and autologous immune serum from co-culture to treat refractory solid tumor.” ASCO Congress, Atlanta, Ga., U.S.A., 1999. The decision of a naive CD 4 T cell to differentiate into either a Th-1 or Th-2 effector is strongly dependent on the cytokine environment, as well as on the density of the ligand to which the T cell responds—affecting, for example, the degree of ligation of the TCR.

The majority of T cells die a short time following activation, thus providing a mechanism of immune response limitation. Certain cells, however, enter a memory pool such that the secondary response is much more rapid than the primary response. The maintenance of memory is complex, yet critical to the cytokine modulator effect, hence the evaluation of the efficacy of treatment through a ninety-day therapeutic window. Different mechanisms of memory through this complex relationship and its phases influence how long antigen persists after initial challenge and explain conflicting results of various trials addressing the requirement for persistence of antigen for the maintenance of therapeutic effect.

In contrast, B-lymphocytes respond to native, polyvalent antigen and proliferate into antibody producing cells unrestricted by self-recognition. Optimal function of antibody production to monovalent antigens or small peptides often requires T-cell assistance [especially CD4+]. Only those cells rich in MHC Class II gene products are capable of presenting antigen to CD4+ cells, and cell lysis often requires complement system activation. Interferon-gamma [IFN-g] is associated with enhanced MHC Class II gene product expression.

Most cell types in association with MHC Class I gene products can present antigen to CD8+ T-lymphocytes. Many of these cells are directly cytotoxic, and cytokine production is pronounced. Interferon-alpha [IFN-a] down-regulates B-cell antibody synthesis but enhances cytotoxic CD8+ cell activity. IFN-a and IFN-g are synergistic. IL-2 augments this process and permits expansion of the immune response.

Production of these cytokines is enhanced by heat, which also stimulates cytotoxic cell activity unrestricted by self-recognition. Pontiggia, P, and Mathe, G, “A new mode of cancer cell death induced by hyperthermia and nonspecific (macrophagic) cancer immunotherapy: lysosomal exocytosis,” Biomed. Pharmacother. (1994) 49: 54. A by-product is a heat shock protein, which induces apoptosis of autologous tumor. Tamura, T, Peng, P, Liu, K, Daou, M, Srivastava, P K, “Immunotherapy of tumors with autologous tumor derived heat shock protein preparations,” Science (1997) 278: 117-120.

The process continues as lysis of target cells triggers complement activation and sustains cytotoxic cell activity perpetuating that process. Lymphocyte response, however, is down-regulated by continuous exposure to high doses of cytokines, which may also lead to organ damage as other cells are recruited as targets and effectors. Continuous exposure also leads to apoptosis of lymphocyte clones. In 1994, Alonso reported six complete remissions and twelve partial remissions in ninety-one patients with metastatic solid tumor refractory to usual therapies who were treated by an intermittent immunotherapy schedule. Alonso, K, and Medenica, R, “Immunomodulation in the treatment of malignant disorders,” Am. J. Clin. Oncol. (1994) 17: 41-44. Many patients later responded to chemotherapy agents. Responses from immunotherapy in published literature endure for months only.

Limitations of autologous tumor vaccines have been well reviewed. Ravindranath, M H, and Morton, D L, “Immunotherapy with vaccines,” in Holland, J F, Bast, R C, Morton, D L, Frei III, E, Kofe, D W, Weichselbaum, R R (eds.), Cancer Medicine (4th edition). Baltimore. William and Wilkins. 1997. Pp 1179-1198. Clinical trials of whole cancer cell vaccines and tumor associated antigen vaccines with and without concomitant adjuvant use have been disappointing in the treatment of patients with refractory solid tumors because there was either inadequate or inaccurate cytokine stimulation.

However, the particular and individual immune response to the cancer can be enhanced when autologous lymphocytes are stimulated to produce a range of cytokines which can then be given back to the donor on an intermittent basis. Additionally, if those cells are grown in vitro with a sample of the cancer itself, those cells produce a gene product specific for the cancer and highly effective in the induction of tumor apoptosis. Medenica, R, and Alonso, K, “Production of patient antitumor specific interferon (tentatively named IFN-Pi),” Blood (1987) 70 (Suppl.): 112. Anti-viral properties have also been described with that product. Alonso, K, Pontiggia, P, Medenica, R, Rizzo, R, “Isolation of a 17 kD protein in autologous stimulated serum with potent anti-HIV properties. Description and clinical use,” So. Med. J. (1996) 89: S141.

The co-cultured tumor cells can then be killed, disrupted with ultrasonication, and returned to the patient as an autologous vaccine to direct more precisely the particular and individual response to the cancer. This process has been described by Alonso. Rizzo, R, Silvotti, S, Martano, P, Santamaria, L, Pontiggia, P, Pontiggia, M, Alonso, K, “Autologous primary tumor cultures for adoptive specific antitumoral immunotherapy,” 5th International Conference of Anticancer Research, Corfu, Greece (October 1995). Concomitant use of stimulated autologous immune serum and autologous tumor vaccine have produced sustained responses.

The article of Alonso, K, Page, R, Kindness, G, McLaren, J, “Concomitant use of autologous tumor vaccine and autologous stimulated immune serum in the treatment of refractory solid tumor,” ESMO Congress, Athens, Greece (November 1998), reported the results of treatment of patients with disseminated disease from solid and hematologic tumors with autologous serum [produced in Germany] in conjunction with psychological support and other [marginal] therapies. 41 patients were treated and were evaluable [8 were lost to follow-up], 4 showed significant tumor response [partial regression]. There were no complete responses. Thirteen patients remained stable during the 90 day follow-up period. Twenty-four progressed.

In 1998, in an unpublished study, Page completed a toxicity and dosing experimental study using autologous vaccine and serum in a similar group of patients with disseminated solid tumors. Of the sixteen patients who were treated and evaluable, no patients were lost to follow-up, and three patients showed significant tumor response [partial response], but no complete responses. Seven remained stable over a 90 day period of follow-up. Four progressed. Two showed mixed responses and were biopsied for the preparation of additional vaccine. The responses from the administration of serum and vaccine were significantly better than from the use of serum alone [p<0.01]. No adverse reactions [National Institute of Health Common Toxicity Criteria Grade 1 or greater] were described. The study examined two methods of administration—concomitant thrice weekly vaccine/serum administration and monthly vaccine with thrice weekly serum administration -and found no obvious difference in toxicity or efficacy. [Though the numbers employed in each arm are both small, the combined preparation for thrice weekly administration is considerably simpler to deliver.]

The literature also suggests that any deficiency in cystine steers inflammatory response toward a Th-2 response, rather than the more desirable Th-1 response. Thus, in the context of this invention, cystine is also recommended because, in addition to its anti-inflammatory benefits, which per se have efficacy, the Th-1 response is also enhanced relative to the Th-2 response. Peterson, J. et al, “Glutathione levels in antigen-presenting cells modulate Th1 versus Th2 response patterns,” Vol 95(6), Proceedings Nat'l Acad. Sci. USA p. 3071-76 (Mar. 17, 1998).

### OBJECTIVES OF THE INVENTION

The purpose of the invention is to deploy the body's immune system to facilitate the partial or total destruction of a malignancy. By harvesting of memory T cells, virgin T cells, and dendritic cells, immune response to cancer can be enhanced by stimulating the autologous lymphocytes to produce a range of cytokines which are then given back to the donor on an intermittent basis. By growing the cancer cells in vitro with the white blood cells, the cells produce a composition specific for the cancer. The invention proposes a composition in which the tumor cells and white blood cells are challenged with phytohemagglutin (“PHA”). After washing, in a step not previously combined in the literature or practice, the culture is subjected to heat shock to generate additional cancer fighting compounds. The overall objective is to use cultured white blood cells, with particular attention to purity, stimulated to form an autologous tumor-specific cytokine modulator, to increase the anti-tumor response of a patient using adjuvant therapy in the treatment of cancer. The cytokine modulator is then highly effective in the induction of tumor apoptosis. Evaluation of the product prior to administration is also possible utilizing a preferred tableau of measures, but at least examining IL-2 and IL-8. Moreover, for patients with severely compromised immune systems, the invention proposes to use a compatible blood type from a similar mammalian species, preferably the same species, to be combined with the tumor cells according to the invention to hopefully, in conjunction with cystine, as defined, augment the patient's life expectancy. In all events, the therapy is intended to not further diminish the patient's quality of life such as a course of traditional chemotherapy or radiation therapy often does.

The invention also contemplates the ability to evaluate efficacy not only of the proposed cytokine modulator, but any cytokine modulator and for that matter any cancer therapy. This can be done by examining white cell response in vivo with respect to a cancer therapy for a selected tumor or tumors, or to evaluate in an early decision fashion the results of an in vitro therapy through application of the procedures applicable to the cytokine modulator. Essentially key factors, at least IL-2 and IL-8, and the tableau set out are viewed. This has been an area in which only time-consuming and lengthy trials have been the means of evaluation. The invention also enables the more accurate evaluation of efficacy of a treatment by consideration of certain important variables for multi-parameter testing. Thus, an immune-compromised patient may not respond to a treatment, but a non-immune compromised patient will respond. The invention enables hope for immune compromised patients by proposing to use the patient's tumor and compatible white blood cell fractions from a non-compromised patient. Testing as discussed in the invention enables early evaluation of the probability of success of the proposed course of treatment. Cystine provides a non-invasive, and quality of life preservation method to augment a patient's immune system.

Cystine is used to enhance anti-inflammatory response, and to steer inflammatory response toward a Th-1 response.

### DESCRIPTION OF THE INVENTION

Preferred Patient Base for Preferred Mode of Invention:

The preferable mode of invention is preferably used on an upper class of patients with solid tumor with a Karnofsky performance status of 70% or above, meaning a patient who is normal, no complaints; is able to carry on normal activities; has only minor signs or symptoms of disease, or cares for self but unable to carry on normal activity, as opposed to a middle class of patients who require occasional assistance, who require considerable assistance and frequent medical care, who are disabled and require special care and assistance, and as further opposed to a lower class of patients who are severely disabled with hospitalization indicated though death not imminent; very ill with hospitalization and active supportive treatment necessary, or those who are moribund.

Unfortunately, those in the lower class tend to have such severely compromised immune systems that they have substantially less tendency to be responsive to the invention. The middle class often reflects substantial compromise of the immune system.

Other measures of the preferred patient base for the preferred mode of the invention are life expectancy greater than 3 months; liver transaminases less than twice the upper limit of normal; the following biochemistry: Granulocyte>1,000/fl and W[hite]B[lood]C[ell]>3,000/fl; platelet count>100,000/fl; hematocrit>30% or Hemoglobin>10.0 g/dl; serum creatinine<2.0 g/dl; total Bilirubin in the normal range and SGPT (ALT), 5.0×upper limit of normal. In addition, Left Ventricular Ejection Fraction (“LVEF”) within the normal range and no evidence of contractility disturbance on echocardiography is preferred. It is preferred there be no recent history [6 months] of stroke, heart attack, or peripheral vascular disease. The FEV should be greater than 80% of expected for age and body habits. Further, the Prothrombin Time and Activated Partial Thromboplastin Time should be within the normal range.

Patients with the following characteristics are less likely or not likely to respond: Patients with fully compromised immune systems; pregnant women (women should preferably have adequate contraception); and heart classification III or IV according to the following table:

| | Cardiac/ | | | | |
|---|---|---|---|---|---|
| | Physical | | | Need for | Physical |
| | ability | | Limita- | Additional | ability to |
| Class | impairment | Symptoms | tions | Rest | work |
| I | None | None | None | None | Full time |
| II | Moderate | Slight | Slight | Occasional | Usually full |
| III | Moderate | Some | Less than | Marked | Part-time |
| | | | ordinary | | |
| | | | activity | | |
| IV | Severe | May be | Extreme | Marked | Unable to |
| | | present | | | work any |
| | | even at | | | activity |
| | | rest | | | increases |
| | | | | | discomfort |

Other patients who are less likely or not likely to respond include those with Myocardial infarction, stroke, or arterial surgery within six months of evaluation for cytokine modulator administration; Abnormal laboratory findings of serum creatinine>3.0 g/dl, or total bilirubin>2.5 mg/dl, or SGPT (ALT)>6.0×upper limit of normal; History of angioedema or of allergic reactions to any agent in the invention; Central nervous system bleeds attributable to a bleeding diathesis, thrombocytopenia, or a previous cerebrovascular accident; Space occupying brain lesions; and Patients involved with illegal drug use.

Prior to treatment, the preferred mode recommends certain examinations and tests set out in Addendum 3 [Addenda numbered 1 and 2 are not used]. Response to the cytokine modulator is normally expected within 90 days. The inventors also recommend establishment of each of the items in Tables 1,2,3 ,4,VIIIA and VIIIB.

In order to craft a more careful response to a cancer diagnosis and tailor therapy, the inventors propose monitoring peroxidation products by measuring the isoprostanes. Isoprostanes are prostaglandin-like compounds which are formed by free radical catalysed peroxidation of arachidonic acid esterified in membrane phospholipids (Neurochem Res October 2000; 25(9-10):1357-64). This is one mechanism for the damage from excess arachidonic acid that may be seen with the use of the selective COX-2 inhibitors and contributes to explaining the toxic effect of a selective COX-2 inhibitor, especially in rapidly dividing cells. However, presence of the isoprostane in the blood or urine would signal an upper limit has been reached of the COX-2 inhibitor above which the risk of kidney or liver damage may increase.

The systemic effects of the isoprostanes should be monitored by following the plasma and urinary excretion of the major oxidation products such as levels of 8-iso-prostaglandin F(2alpha) (8-iso-PGF(2alpha)), a major isoprostane, and of 15-oxo-dihydro-PGF(2alpha), a major metabolite of PGF(2alpha), as indicators of non-enzymic and enzymic arachidonic acid oxidation (collectively referred to as isoprostanes). Using the isoprostane levels as indicators the treatment dose of the COX-2 inhibitor can be maximized to give the maximum tolerated dose for use in cancer therapy without creating excessive systemic toxicity. More lipid oxidation activity indicates more oxidation stress usually characteristic of cancer activity. A long-term falling level of isoprostanes will mean for COX-2 expressing cancers that there is relatively less cancer risk. An ELISA test for isoprostate level is available from Cayman Chemical Company, 11800 E. Ellsworth Rd., Ann Arbor, Mich.

The meaning and definition of Cyclooxygenase-2 inhibitor (“COX-2 inhibitor”) in this invention shall include the following: all of the compounds and substances beginning on page 8 of Winokur WO 99/20110 as members of three distinct structural classes of selective COX-2 inhibitor compounds, and the compounds and substances which are selective COX-2 inhibitors in Nichtberger, U.S. Pat. No. 6,136,804, Oct. 24, 2000, entitled “Combination therapy for treating, preventing, or reducing the risks associated with acute coronary ischemic syndrome and related conditions”, and the compounds and substances which are selective COX-2 inhibitors in Isakson et al, PCT application WO/09641645 published 27 Dec. 1996, filed as PCT/US 9509905 on 12 Jun. 1995, entitled “Combination of a Cyclooxygenase-2 Inhibitor and a Leukotriene B4 Receptor Antagonist for the Treatment of Inflammations.” The meaning of COX-2 inhibitor in this invention shall include the compounds and substances referenced and incorporated into Winokur WO99/20110 by reference to art therein, the compounds and substances referenced and incorporated into Nichtberger, U.S. Pat. No. 6,136,804, Oct. 24, 2000, by reference to art therein, and the compounds and substances which are COX-2 inhibitors referenced and incorporated into Isakson et al, PCT application WO/09641645 published 27 Dec. 1996, filed as PCT/US 9509905 on 12 Jun. 1995, entitled “Combination of a Cyclooxygenase-2 Inhibitor and a Leukotriene B4 Receptor Antagonist for the Treatment of Inflammations.” The meaning of COX-2 inhibitor in this invention also includes rofecoxib, and celecoxib, marketed as VIOXX and CELEBREX by Merck and Searle/Pfizer respectively. Rofecoxib is discussed in Winokur, WO99/20110 as compound 3, on p.9. Celecoxib is discussed as SC-58635 in the same reference, and in T. Penning, Synthesis and biological evaluation of the 1,5-diarylpyrazole class of cyclooxygenase-2 inhibitors: identification of 4-[5-(4-methylphenyl)-3-(trifluoromethyl)-1H-pyrozol-1-yl]benzenesulfonamide (SC-58635, celecoxib)”, J. Med. Chem. 1997 Apr. 25: 40(9): 1347-56. The meaning of COX-2 inhibitor in this invention also includes SC299 referred to as a fluorescent diaryloxazole. C. Lanzo et al, “Fluorescence quenching analysis of the association and dissociation of a diarylheterocycle to cyclooxygenasel-1 and cyclooxygenase-2: dynamic basis of cycloxygenase-2 selectivity”, Biochemistry 2000 May 23 vol. 39(20):6228-34, and in J. Talley et al, “4,5-Diaryloxazole inhibitors of cyclooxygenase-2 (COX-2)”, Med. Res. Rev. May 1999; 19(3): 199-208. The meaning of COX-2inhibitor in this invention also includes valdecoxib, See, “4-[5-Methyl-3-phenylisoxazol-1-yl]benzenesulfonamide, Valdecoxib: A Potent and Selective Inhibitor of COX-2”, J. Med. Chem. 2000, Vol. 43: 775-777, and parecoxib, sodium salt or parecoxib sodium, See, N-[[(5-methyl-3-phenylixoxazol-4yl)-phenyl]sulfonyl]propanimide, Sodium Salt, Parecoxib Sodium: A Potent and Selective Inhibitor of COX-2 for Parenteral Administration”, J. Med. Chem. 2000, Vol. 43: 1661-1663. The meaning of COX-2 inhibitor in this invention also includes the substitution of the sulfonamide moiety as a suitable replacement for the methylsulfonyl moiety. See, J. Carter et al, Synthesis and activity of sulfonamide-substituted 4,5-diaryl thiazoles as selective cyclooxygenase-2 inhibitors”, Bioorg. Med. Chem. Lett 1999 Apr. 19:Vol. 9(8): 1171-74, and compounds referenced in the article “Design and synthesis of sulfonyl-substituted 4,5-diarylthiazoles as selective cyclooxygenase-2 inhibitors”, Bioorg. Med. Chem. Lett 1999 Apr. 19:Vol. 9(8): 1167-70. The meaning of this invention includes a COX-2 inhibitor, NS398 referenced in two articles: Attiga et al, “Inhibitors of Prostaglandin Synthesis Inhibit Human Prostate Tumor Cell Invasiveness and Reduce the Release of Matrix Metalloproteinases”, 60 Cancer Research 4629-4637, Aug. 15, 2000, and in “The cyclooxygenase-2 inhibitor celecoxib induces apoptosis by blocking Akt activation in human prostate cancer cells independently of Bcl-2,” Hsu et al, 275(15) J. Biol. Chem. 11397-11403 (2000). The meaning of COX-2 inhibitor in this invention includes the cyclo-oxygenase-2 selective compounds referenced in Mitchell et al, “Cyclo-oxygenase-2: pharmacology, physiology, biochemistry and relevance to NSAID therapy”, Brit. J. of Pharmacology (1999) vol.128: 1121-1132, see especially p. 1126. The meaning of COX-2 inhibitor in this invention includes so-called NO-NSAIDs or nitric oxide-releasing-NSAIDs referred to in L. Jackson et al, “COX-2 Selective Nonsteriodal Anti-Inflammatory Drugs: Do They Really Offer Any Advantages?”, Drugs, June, 2000 vol. 59(6): 1207-1216 and the articles at footnotes 27, and 28. Also included in the meaning of COX-2 inhibitor in this invention includes any substance that selectively inhibits the COX-2 isoenzyme over the COX-1 isoenzyme in a ratio of greater than 10 to 1 and preferably in ratio of at least 40 to 1 as referenced in Winokur WO 99/20110, and has one substituent having both atoms with free electrons under traditional valence-shell-electron-pair-repulsion theory located on a cyclic ring (as in the sulfylamine portion of celecoxib), and a second substituent located on a different ring sufficiently far from said first substituent to have no significant electron interaction with the first substituent. The second substituent should have an electronegativity within such substituent greater than 0.5, or the second substituent should be an atom located on the periphery of the compound selected from the group of a halogen F, Cl, Br or I, or A group VI element S or O. Thus for purposes of this last included meaning of a COX-2 inhibitor, one portion of the COX-2 inhibitor should be hydrophilic and the other portion lipophilic. Also included as a COX-2 inhibitor are compounds listed at page 553 in Pharmacotherapy, 4^{th} ed: A Pathophysiologic Approach, Depiro et al (McGraw Hill 1999) including nabumetone and entodolac. Recognizing that there is overlap among the selective COX-2 inhibitors set out in this paragraph, the intent of the term COX-2 inhibitor is to comprehensively include all selective COX-2 inhibitors, selective in the sense of inhibiting COX-2 over COX-1. The package inserts for rofecoxib and celecoxib are attached and adopted herein by reference. The inventors add to the class of COX-2 inhibitors useful in the invention the drug bearing the name etoricoxib referenced in the Wall Street Journal, Dec. 13, 2000 manufactured by Merck. The inventors also include as a selective COX-2 inhibitor the flavonoid antioxidant silymarin which demonstrated significant COX-2 inhibition relative to COX-1 inhibition. The silymarin also showed protection against depletion of glutathione peroxidase. Zhao et al, Mol. Carcinog. December 1999, Vol 26(4):321-33 PMID 10569809. Silymarin has been used to treat liver diseases in Europe.

The term COX-2 inhibitor includes all pharmaceutically acceptable salts for the COX-2 inhibiting compound selected. Examples of such salt forms of COX-2 inhibitors include but are not limited to salts derived from inorganic bases including aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N-dibenzylethylenediamine, diethylamide, 2-diethylaminoethanol, 2-dimethylaminoethanol, ethanolamine, ethylenediamine, N-ethylmorpholine, -ethylpiperidine, glutamine, glucosamine, histidine, hydrabamine, isopropylamine, lysine, methyglucamine, morpholine, piperazine, piperidine, polyamine resins, procaine, purine, theobromine, triethylamine, trimethylamine, tripropylamine, troethamine, and the like.

Preferred Mode of Composition and Manufacture of the Invention:

The strategy for the cytokine modulator is the deployment of the immune system to facilitate the destruction of a malignancy.

If memory T cells, virgin T cells, and dendritic cells are harvested, immune response to cancer can be enhanced by stimulating the autologous lymphocytes to produce a range of cytokines which are then given back to the donor on an intermittent basis. Additionally, if those cells are grown in vitro with a sample of the cancer itself, those cells produce a gene product specific for the cancer and highly effective in the induction of tumor apoptosis.

The development and delivery of an effective cytokine modulator preferably uses an efficient but sterile system for the collection, isolation, and harvesting of the optimal number of cells. Mononuclear cells can be harvested by deploying a sterile evacuated tube containing trisodium citrate. The cell separation medium is comprised of a polyester gel and a density gradient liquid which is a thixotropic medium. Thixotropic medium enables finer distinction in cell separation because both cell density and cell size factor into gradient separation. This configuration permits cell separation during a single configuration step. The separated sample can then be transferred under sterile conditions rom the tube, thus maintaining the sterility and integrity of the cell layers. Otherwise, more traditional means such as a Sefedex column can be deployed.

Once activated by antigen or appropriate antigen-presenting cells, the T cells acquire an effector function. The concentration of these cells enhances cytokine production in the final cytokine modulator product. Indeed, virgin T cells are activated by stimulation of the TCR with antigen—MCH. These surface interactions initiate a complex cascade of signal transduction events in the culture medium, which result in cell division and acquisition of effector finction. The helper T cells can be divided into two distinct subsets, based on the profile of cytokine expression. Th 1 cells produce IL-2, INF-g, ECP and TNF while Th 2 cells produce IL-4, IL-5, IL-6, IL-10 and IL-13. The differential pattern of cytokine expression underlies functional differences between subsets which may play an important role for the effectiveness of a particular cytokine modulator in the treatment of advanced disease as well as the cytokine effect on micrometastases. IL-10 may be measured as a corollary to IL-6 as to downregulation of Th 2 response.

Thus, Th-1 cells are inflammatory cells, which induce cellular immunity such as enhanced macrophage activity, whereas TH-2 cells act to support B cell in production of antibody. The cytotoxic cells exhibit a Th-1 profile of cytokine secretion, a theory supported by previous work. Alonso, K, “Interleukin-2 with immunomodulation in the treatment of human malignancy: a pilot study,” So. Med. J. (1988) 81 (Suppl. 4): 43.

### CHEMISTRY AND MANUFACTURING DATA

Patients' blood, tumor biopsy, and cytokine modulator are clearly labeled from the time of cell collection, through cytokine modulator production and shipment, until final administration of the cytokine modulator to ensure that each patient receives the correct final product.

### PRODUCT PREPARATION

1) A sterile Becton-Dickinson tube has placed in it

A) trisodium citrate,

B) a density gel more technically referred to as a polyester thixotropic gel with density gradient liquid medium of Ficoll [Hypaque solution]400 (a thixotropic medium), and

C) sodium metrizoate or sodium distrizoate solution.

The preferred specific quantities and order are:

a) 0.45 mL of 0.1 M sodium citrate (top fluid layer)

b) 1.8 gm Polyester gel (middle layer)

c) 1.0 mL polysaccharide/sodium diatryoate solution (Ficoll Hypaque solution; bottom fluid layer)

d) Silicone coated Becton-Dickinson glass tube

e) Silicone coated rubber stopper

2) The tube is otherwise evacuated.

3) Whole blood is collected under the supervision of a physician by phlebotomy, normally at a remote location into the evacuated tube referenced in steps 1 and 2 which tube contains the appropriate cell separation medium, resulting in an anticoagulated sample being shipped. The tube is not intentionally shaken or mixed but will have a natural separation gradient which will be enhanced by centrifugation.

4) The tube is shipped by overnight delivery and ambient temperature with the later-referenced tumor tissue.

5) Centrifugation 20 minutes at 1500 to 1800 RCF (Relative Centrifugal Force) where r (cm) is the radial distance from the centrifuge center post to the tube bottom, when the tube is in the horizontal position.

6) One key to the preferred mode of this invention is the careful separation and isolation of WBC's in a Becton-Dickinson tube that has a density gel to produce a separation gradient with the R[ed]B[lood]C[ell]'s passing through the gradient and the WBC's on top of the gradient. This preparation is then centrifuged to isolate the mononuclear cells above the medium. The use of a thixotropic solution enables finer distinction in cell separation because both cell density and cell size factor into gradient separation.

7) The WBC's are harvested by pipetting from above the liquid interface.

In sum, the Ficoll Hypaque density fluid and a polyester gel barrier separates the two liquids. The result is a single tube system for the collection of whole blood and the separation of mononuclear cells, reducing the risk of sample contamination and eliminating the need for additional tubes, pipettes and reagents. The patient's sample can be transported without removal from the tube, thus maintaining the sterility and integrity of the transport environment with the gel forming a stable barrier between the cell layers. Due to the relative low density of mononuclear cells in relation to whole blood, separation is achieved.

8) The levels of cytokines in the WBC's referred to in paragraph 7 are determined and recorded. One unique aspect of this invention is the ability to predict the likelihood of efficacy of the proposed cytokine modulator by reviewing whether cytokine levels are higher in the composed product than in the initially harvested and separated product. The use of thixotropic solution for separation facilitates more accurate assessment of the cytokine levels. Pretesting of harvested cells through SMAC profile, liver profile, and immunological tests will provide a baseline of cytokine levels for each patient prior to application of this invention. Comparisons will made between this baseline and the results of identical tests on the completed product to decide whether the patient should receive the cytokine modulator. Cytokine levels in the harvested cells should be raised above normal range (to be determined individually for each patient) by the composition or method in this invention before cytokine modulator is administered. If there is insufficient cytokine level, then the cytokine modulator is preferably not used until a higher-than-pre-stimulation cytokine level is achieved. The principal cells harvested will include CD 4, CD 7 (NK), Dendritic cells and Naive CD4, CD 6, T and B cells, CD 19 -21, B cells, CD 14—monocytes/macrophages, and NK cells.

9) Contemporaneously to the harvest of whole blood from the patient is a harvest of live cancer tumor from the patient. Tumor is obtained as a needle biopsy, surgical biopsy, or as malignant cells in an effusion. At all times aseptic technique is employed in the harvesting of tissue.

10) The patient's serum is aliquoted and assayed for cytokines prior to PHA stimulation. All baseline levels along with stimulated cytokine response become part of the respective product reporting (description) of the final product.

11) The cancer cells are transported to the laboratory in physiologic saline or in tissue culture under ambient conditions. The tumor may be stored may also be kept at 4 degrees Centrigrade. On arrival at the laboratory, tumor cells undergo pathological microscopic examination and staging. Large amounts of tissue are macerated under laminar flow conditions class 100,000, aseptically, with tissue then collected in DMEM F12. The milieu largely consists of single tumor cells though microscopic cell clumps.

12) Macerated tumor cells will be challenged in the presence of harvested dendritic lympohocyte, natural killer, macrophage and platelet rich plasma.

13) The mixture from step 7 is then transferred to a sterile culture vessel, referred to as an incubation flask, containing DMEM F 12 (Sigma Laboratories, Inc.) and the macerated effusion of cancer cells is dispersed into the incubation flask. This mixture is stimulated with PHA 5mg/mL sterile stock PHA solution which has been filtered through a 0.22 μMF filter (PHA available from Sigma Chemical Co., St. Louis, Mo. for six (6) hours. Following incubation at 37° C. for one hour, the milieu is then clarified by centrifugation and washed in 0.9%(w/v) sterile saline three times with ten (10) minute centrifugation steps. This process of washing ensures the removal of PHA from the culture media. The washed mixture is placed placed under sterile conditions into a 100 mL DMEM f12 solution.

14) The mixture temperature is raised to 42° C. for one hour to stimulate heat shock proteins and then incubated at 37° C. for at least 96 hours and preferably 120 hours.

15) The result is a supernatant which is collected and clarified by centrifugation, 600×g (gravitational constant) for ten (10) minutes prior to filtration through a 0.22 micron millipore filter.

16) The cytokine modulator is then evaluated for immune system markers, preferably: IL-2, IL-6, IL-8, IL-10, IL-12, TNF, IFN, ECP. The expected levels are outlined in Table I. The filtered supernatant is also evaluated for: endotoxin, mycoplasma, and sterility testing.

17) Following sterility and quality control evaluation, the cytokine modulator is aseptically dispensed into 1.0 mL sterile single dose ampules.

18) An aliquot of the cytokine modulator is stored at −76C for comparison with follow-up samples and laboratory testing.

### LABELING AND SHIPMENT OF SAMPLES

All serum samples should be properly labeled and sent by expeditiously handling, preferably by air express.

All tumor biopsy material should be properly labeled and shipped in physiologic saline or in tissue culture medium to the central laboratory by air express.

Cytokine Modulator PACKAGING AND SHIPMENT

The cytokine modulator should be packaged in sterile, 1 mL, single-dose vials.

Preferred Mode of Administration to Patient

The cytokine modulator is administered over a ninety-day period as a series of single 1 ml doses intradermally three times per week.

Schedule of Cytokine modulator Administration:

Three times a week of a. therapeutically effective amount for ninety days (Monday, Wednesday, Friday).

Dosage will normally consist of 1 ml of Cytokine modulator. This dosage is recommended as a guide for use in the refractory subject.

Table I sets forth the name and reference ranges and a background of function of various cytokines and markers as to immune and anti-cancer function. Periodic comparison with the baseline numbers established prior to treatment referenced in Tables I, 2,3,4, VIIIA and VIIIB is appropriate to monitor progress. Glutathione levels and antioxidant capacity should increase to normal levels.

TABLE I
CYTOKINE PROFILE AND RANGES

| Test/ | FactorName/range | Brief description |
|---|---|---|
| GSH | Glutathione | RBC, WBC,plasma-GSH represents a significant pool for |
| | 200-400 μmole/L | homeostatic immune function and cytokine regulation |
| AOA | Antioxidant capacity | Measurement of the total antioxidant capacity of the organism |
| | 0.95-1.6 mmole/L | to quench free radicals and eliminate inflammatory processes |
| IL-2 | Interleukin-2 | Activates lymphocytes, potent stimulator of cytokine activated |
| | 0.0-4.0 pg/mL | killer cells (LAK's) which demonstrate enhanced MHC |
| | | non-restricted cytotoxicity. Used for renal cell CA-encourage |
| | | Tc1 activity |
| IL-6 | Interleukin-6 | Involved in T-cell activation; in nesting cells induce the expression |
| | 0.0-149 pg/mL | of receptors for T-cell growth factor. Very important in inducing |
| | | B-cells to differentiate into antibody-forming cells. In liver, it stimulate |
| | | production of acute phase proteins. Growth factor for multiple myelom |
| IL-8 | Interleukin-8 | Proinflammatory cytokine released from range of cells including |
| | 0.0-70 pg/mL | monocytes, endothelial cells, epithelial cells, hepatocytes, fibroblasts and |
| | | chondrocytes |
| IL-12 | Interleukin-12 | Potent initial stimulus for T-and Nk-cell, IFN(IFN = interferon)-γ |
| | Range 0.7 pg/mL-7000 pg/mL | production. May encourage Tc1 generation. Potentiates NK cell |
| | | to release IFN-8. Works in a manner complementary to IL-10; |
| | | increase in level compared to baseline indicates potential for increased c |
| | | mediated response |
| TNF | Tumor Necrosis Factor | Activates macrophage (mφs) and neutrophils |
| | 0.0-4.9 pg/mL | |
| IFN-γ | Interferon-gamma | Encourages Tc1 generation role in early phase of immune |
| | 0.0-1.5 pg/mL | response including antiviral and antiproliferative properties |
| IFN-α | Interferon-alpha | Induces IL-2 and can be used to switch Th cells from a Th2 |
| | 0.0-1.5 pg/mL | to a Th1 profile |
| ECP | Eosinophilic cationic protein | Potent indicator of eosinophilic degranulation resulting in |
| | 1.5-5.5 mg/mL | a wide range of inflammatory conditions: autoimmune disease, |
| | | bronchial asthma, parasitic infections, viral infections |
| IL-10 | Interleukin-10 | Potent blocker of activation of cytokine synthesis and several |
| | ED₅₀ = 0.5 ng-1 ng/mL | accessory functions of macrophages; produced in CD4+ T cells |
| | | and T cell clones, and other cells; downregulation indicates lessened |
| | | interference with cytokine synthesis of cytokines needing upregulation |
| | | and lessened macrophage activity interference |

Administration by health professional:

The Cytokine modulator should be routinely administered intradermally at separate sites per injection schedule. The preferred mode of administration is shipment directly to licensed physicians and administration by physicians of the product to patients preferably in single dose vials. Each single dose vial should be inspected visually for extraneous particulate matter prior to administration as a final check on contamination or effectiveness. If this condition exists, the Cytokine modulator should NOT be used.

The Cytokine modulator should be shaken well before withdrawing each dose to obtain a homogeneous suspension. The Cytokine modulator should not be injected into the gluteal area or areas where there may be a major nerve trunk.

Prior to injection, the skin over the injection site should be cleansed with a suitable germicide. After the needle is inserted, it should be aspirated to ensure that it has not entered a blood vessel.

The preferred mode of the invention also proposes to augment the immune system competency by the administration of cystine prior to commencement of treatment after a Baseline blood sample has been taken. The addition of cystine, cysteine, N-acetyl cysteine, or the pharmaceutically acceptable salt of those substances yields another effect in this invention not facially evident from the independent properties of the basic components of the invention (hereafter each substance or a pharmaceutically acceptable salt is referred to as a “cystine family member” Cancer cells function with a much higher ratio of anaerobic to aerobic function than do normal cells, almost to the point of “anaerobic function”. The glutathione cycle is a critical body cycle whose importance has not been fully appreciated. Administration of a cystine family member, preferably cystine, which has the best and most rapid upload into the glutathione pathway, or N-acetyl cysteine, enhances the immune system competency of the cancer patient. Because normal body cells have a relatively aerobic function highly dependent on glutathione, the enhancement of the glutathione pathway has a disproportionately beneficial effect on normal cells than enhancement of the glutathione pathway in a cancer cell.

Cystine is (3,3′-dithiobis [2-aminopropanoic acid]). Cystine is readily reduced to cysteine. Cystine is present in most mammalian hair and keratin.

Cysteine is 2-amino-3-mercapto propanoic acid. It is readily converted by oxioreduction to cystine. It is a constituent of glutathione and abundantly present in the metallothioneines.

Cystine in the body-useful form as L-cystine is available from Spectrum Chemical Mfg. Corp. 14422 S. San Pedro St., Gardena, Calif. 90248.

Cystine, cysteine, and N-Acetyl cysteine and pharmaceutically acceptable salts, including the pharmaceutically active forms described in Kozhemyakin et al, published by WIPO as WO 00/031120, PCT/RU99/00453, filed internationally on 19 Nov. 1999, “Hexapeptide with the Stabilized Disulfide Bond and Derivatives Thereof Regulating Metabolism, Proliferation, Differentiation and Apoptosis,” will all collectively be referred to as cystine in this invention. Included in the term cystine is also any therapeutically beneficial sulfur donating compound, including ebselen, which interacts with the glutathione pathway. The invention contemplates in the term cystine undenatured whey protein products designed to have enhanced cystine concentration as well as protein products which contain cysteine and cystine. They can be in the form of food products.

The addition of cystine, cysteine, N-acetyl cysteine, or the pharmaceutically acceptable salt of those substances yields another effect in this invention not facially evident from the independent properties of the basic components of the invention (hereafter each substance or a pharmaceutically acceptable salt is referred to as a “cystine”). Administration of a cystine family member, preferably cystine, which has the best and most rapid upload into the glutathione pathway and better storage capability by the body, or N-acetyl cysteine, enhances the immune system competency of the patient. Included in the term cystine is also any therapeutically beneficial sulfur donating compound, including ebselen, which interacts with the glutathione pathway. The invention contemplates in the term cystine undenatured whey protein products designed to have enhanced cystine concentration as well protein products which contain cysteine and cystine. They can be in the form of food products.

Lipoic acid can be an adjunct to the cystine.

Cystine and enhancement of the glutathione level and pathway has a second important and unexpected effect in conjunction with the composition and treatment: The avoidance of a glutathione deficiency steers the patient to have a higher Th-1 response to Th-2 response ratio than the patient would have with any glutathione deficiency. Peterson, J. et al, “Glutathione levels in antigen-presenting cells modulate Th1 versus Th2 response patterns,” Vol 95(6), Proceedings Nat'l Acad. Sci. USA p. 3071-76 (Mar. 17, 1998).

The amount of cystine to be included in the combination is a therapeutically effective amount to reach normal glutathione levels. Such therapeutically effective amount should preferably be in an amount initially of 140 mg/Kg twice per day. Glutathione levels and assay procedure can be seen in Table and Addendum 17.

The invention also proposes that patients be screened for GST defects. Provisional Application 60/227,151 filed by Kindness, Schumm and Walter is incorporated by reference. The significance of a GST defect is that if there is a GST defect, the therapeutically effective amount of cystine may have to be increased. The patient's glutathione level should be evaluated and cystine administered in a therapeutically effective amount to reach normal glutathione levels and those levels monitored weekly until a normal level is achieved at which time testing may be less often.

Follow-up during treatment:

Patients should be examined at least monthly both during the treatment program and quarterly thereafter (to sixty months). Patients should have a clinical history including the date of symptoms, the date of diagnosis, the therapy given, and the results observed with all drugs administered in the previous month will be recorded and all current medications noted. Each patient should be questioned about possible adverse reaction since the last visit, and any reactions described will be recorded on appropriate forms to appropriate regulatory authorities. As medically indicated, blood should be drawn for clinical laboratory tests and imaging studies performed, and for recommended monitoring referenced in this description. Certain test protocols which may not be generally known are attached.

Evaluation of patient:

Treatment is considered successful with a complete response if there is total disappearance of all reversible clinical evidence of disease for at least two measurement periods separated by at least four weeks. Bone lesions will have recalcified and blastic lesions will have become radiolucent, “walled-off”, or of normal density. There is partial response if there is at least a 50% reduction in the size of all measurable tumor volumes as measured by the product of the greatest length and the maximum width and depth; or at least a 30% reduction in unidimensionally measured lesions; or where measurable lesions are non-existent, by a decrease of 50% or more in evaluable disease. The changes must be present in >50% of the involved organ sites. Partial response is not considered to have occurred if lesions may progress or new lesions appear. However, the invention contemplates observation for new cancers that may occur because their presence was initially so small or little that appropriate immune system stimulation could not occur in sufficient concentration. These parameters of response must be present for at least two measurement periods separated by at least four weeks. Progressive disease would be considered to have occurred if there is appearance of a new lesion, or an increase of 25% in the volume of any existing lesion so long as that increase is at least 2 cm². In the case of bone lesions, progressive disease will be assigned after eight weeks of observation of static lesions if there is subjective progressive disease or relapse; subject to the proviso that the new lesion may be a non-apparent initial lesion indicating preparation of a second composition according to this invention. If there is such a new lesion, the invention should be applied to this likely differentiated tumor. Notice that the invention has the capacity to deal with tumors that are not included in the initial tumor sample or to deal with a mutated tumor that becomes non-responsive by repeating the invention's application to such new or mutated tumors.

The patient should be given a quality of life questionnaire to not only capture the medicinal benefits of cytokine modulator, but also to ensure quality of life during and after administration.

### PARAMETERS TO BE FOLLOWED

In order to have efficacious measurement, the following are appropriate prior to commencing the treatment protocol:

Required Laboratory Tests:

CBC and platelet count (to include automated WBC differential)

Biochemical profile (Sodium, Potassium, chloride, creatinine, glucose, Calcium, Phosphorous, total protein, albumin, total bilirubin, SGOT, SGPT, GGT, alkaline phosphatase, cholesterol, triglyceride, uric acid)

Magnesium

HDL-cholesterol, Lpa, APO A, APO B

Ferritin

Pregnancy test (Women Only)

IL-2

IL-6, IL-8, IL-12, INF, TNF, ECP

Urinalysis

Beta-2-Microglobulin

EKG (and Echocardiogram in those patients who have received radiation therapy to the chest, or have a history of heart disease)

Spirometry and PEFR (in those patients who have received radiation therapy to the chest, or have a history of lung disease)

CT or (MRI as appropriate) of brain, chest, abdomen, pelvis, or limb as appropriate to evaluate extent of disease. Bone scan if appropriate.

CEA or CA 15-3 or PSA or CA 125 or other tumor marker as appropriate.

Testing to be repeated monthly during treatment: CBC, biochemical profile, urinalysis, tumor marker, and pregnancy test (women only). Then, quarterly, with lymphocyte subsets and cytokines. Imaging studies will be performed quarterly.

Special attention should be given to detecting adverse effects of cytokine modulator therapy. Abnormal vital signs, any other signs of systemic effects and abnormal laboratory test results should be noted. The physician should note severity of symptoms according to Grade 1-4 (as defined by WHO Toxicity Criteria, Addendum 3) and determine the connection, if any, to the treatment. The cytokine modulator should not be administered if there is Grade 4 hematological toxicity, or Grade 3 non-hematological toxicity—except allergy and alopecia (alopecia of any grade is not considered dose-limiting). Normally, Grade 2 allergic toxicity should also be considered an intolerable adverse reaction.

The treatment should be terminated if there is acceleration of tumor growth, as determined by standard medical evaluation (CAT scan or MRI).

In the best mode of invention, the following procedures should be performed on the cytokine modulator produced for each patient before vials are filled:

Total Protein Assay Procedure (Addendum 11) [prior addenda numbers except Addendum 3 intentionally omitted]

Procedure for Culture Sterility (Addendum 12)

Sterility testing will be performed as outlined in 21 CFR 610.12 for a period of at least 72 hours. The product will not be administered to patients until the results of the sterility assay indicate “no growth.”

LAL Procedure (Addendum 13)

TNF Procedure (Addendum 14)

Mycoplasma (PPLO) Culture Procedure (Addendum 15)

A negative lot release criterion for mycoplasma will be established with results available prior to administration of cytokine modulator to patients. Testing will be performed as outlined in “Points to Consider in the Characterization of Cell Lines Used to Produce Biologicals” (1993).

IFN Procedure (Addendum 16)

Each patient will receive the cytokine modulator for ninety-day periods up to twelve months. Monthly examinations will be required during the treatment program, followed by quarterly examinations up to sixty months. When treatment is completed or when a decision is made to stop treatment, the patient will immediately or within seven days undergo testing and examination shown in the final treatment visit.

The term “therapeutically effective amount” is intended to mean that amount of a drug or pharmaceutical agent that will elicit the biological or medical response of a tissue, a system, animal or human that is being sought by a researcher, veterinarian, medical doctor or other clinician. The term “prophylactically effective amount” is intended to mean that amount of a pharmaceutical drug that will prevent or reduce the risk of occurrence of the biological or medical event that is sought to be prevented in a tissue, a system, animal or human by a researcher, veterinarian, medical doctor or other clinician.

In conclusion, prior to the Addenda, cytokine modulator is preferably used for treating solid tumors that have proven resistant to conventional therapies. For a defined mass, treatment with a cytokine modulator is an important supplement in the preferred mode in addition to surgery which can rapidly excise a defined mass. For less well-defined solid tumors, the therapy is appropriate to avoid physically degrading treatment. The therapy may be used as an adjuvant to chemotherapy after a substantial reduction of tumorous cells. The therapy may be used as an adjuvant to radiation after a substantial reduction of tumorous cells. However, because chemotherapy degrades a patient's immune system more than radiation, the cytokine modulator is likely to be more efficacious as an adjuvant to radiation. A cytokine modulator is a form of immunotherapy, the repairing of the client's own natural immune system so that it can resist tumor cells. Instead of attacking the tumor through external means—surgery, radiation, or chemotherapy—immunotherapy enables the body to control and perhaps eventually rid itself of cancer. The cytokine modulator is a stimulation of the patient's immune system to recognize and destroy tumor cells. Since the cytokine modulator is autologous, and each dosage is made from the patient's own blood and tissue, the inventors believe, and clinical results of similar product have shown various cancers, and with this product for metastatic colorectal cancer, that it retards and effectively mitigates the effects of the patient's own tumor and particular type of cancer.

The invention is not meant to be limited to the disclosures, including best mode of invention herein, and contemplates all equivalents to the invention and similar embodiments to the invention for humans and mammals and veterinary science. Equivalents include all pharmacologically active mixtures, and pharmaceutical equivalents. Equivalents also include cytokines that operate correlatively with other cytokines. For instance, IL-2 is usually upregulated with IL-12, but IL-12 is more commonly measured.

As to the application of the invention and demonstration of safety and efficacy, a patient was described to a physician as having advanced metastatic colorectal cancer that was terminal. The cytokine modulator described in this patent was administered and the patient takes a whey-cystine dietary supplement. The patient has suffered no significant side effects, and the patient has gained weight and maintains weight recovery and outlived with good quality of life his pre-treatment life expectancy.

Previously, another product called Theracine had been produced. The product had some similarities, but a less efficient method of manufacturing, a different set of steps which had no heat shock step and a different resultant product and composition resulted. Seventy-four patients were treated and none died during treatment. Of a group of approximately 45 patients, several of whom did not undergo treatment, 24 patients are known to have ultimately died, though many of them who had stage 4 cancers survived beyond what their life expectancy was without treatment. Many of the rest of this group are alive. Of a group of another 37 patients, several of whom did not undergo treatment, 15 patients are known to have ultimately died, though many of them who had stage 4 cancers survived beyond what their life expectancy was without treatment. Many of the rest of this group are alive.

ADDENDUM 3
WHO Recommendations for Grading of Acute and Subacute Toxicity

| | Grade 0 | Grade 1 | Grade 2 | Grade 3 | Grade 4 |
|---|---|---|---|---|---|
| Blood | | | | | |
| Hemoglobin | >11.0 | 9.5-10.9 | 8.0-9.4 | 6.5-7.9 | <6.5 |
| Leukocyte | >4.0 | 3.0-3.9 | 2.0-2.9 | 1.0-1.9 | <1.0 |
| count | | | | | |
| Granulocyte | >2.0 | 1.5-1.9 | 1.0-1.4 | 0.5-0.9 | <0.5 |
| count | | | | | |
| Platelet | >100 | 75-99 | 50-74 | 25-49 | <25 |
| count | | | | | |
| Hemorrhage | None | Petechiae | Mild blood | Gross blood | Debilitating |
| | | | loss | loss | blood loss |
| GI | | | | | |
| Total | <1.25x | 1.26-2.5x N | 2.6-5.0x N | 5.1-10.0x N | >10.0x N |
| bilirubin | Normal | | | | |
| ALT, AST | <1.25x | 1.26-2.5x N | 2.6-5.0x N | 5.1-10.0x N | >10.0x N |
| | Normal | | | | |
| Alkaline | <1.25x | 1.26-2.5x N | 2.6-5.0x N | 5.1-10.0x N | >10.0x N |
| Phosphatase | Normal | | | | |
| Mouth | No lesions | Erythema | Ulcers; can | Ulcers: | Ulcers; |
| | | | eat solids | liquids only | cannot eat |
| Vomiting | None | Nausea | Transient | Vomiting | Intractable |
| | | | vomiting | requiring | vomiting |
| | | | | therapy | |
| Diarrhea | None | Transient, | Tolerable, | Requiring | Hemorrhage |
| | | <2 days | >2 days | therapy | Dehydration |
| Kidney | | | | | |
| BUN | <1.25x | 1.26-2.5x N | 2.6-5.0x N | 5.1-10.0x N | >10.0x N |
| | Normal | | | | |
| Creatinine | <1.25x | 1.26-2.5x N | 2.6-5.0x N | 5.1-10.0x N | >10.0x N |
| | Normal | | | | |
| Proteinuria | None | 1+ | 2-3+ | 4+ | Nephrosis |
| Hematuria | None | Microscopic | Gross | Clots | Obstruction |
| Lung | no | Mild | Exertional | Dyspnea at | Complete |
| | symptoms | symptoms | Dyspnea | rest | bed rest |
| | | | | | required |
| Fever-Drug | None | <38.0 C | 38.0-40.0 C | >40.0 C | Fever with |
| | | | | | hypotension |
| Allergy | None | Edema | Broncho- | Broncho- | Ana- |
| | | | spasm, no | spasm, | phylaxis |
| | | | parenteral | parenteral | |
| | | | therapy | therapy | |
| | | | required | required | |
| Skin | No | Erythema | Dry with | Moist with | Exfoliative |
| | symptoms | | Desquam- | Desqua- | Dermatitis, |
| | | | ation, or | mation or | Necrosis |
| | | | Vesi- | Ulceration | |
| | | | culation or | | |
| | | | Pruritis | | |
| Hair | No hair loss | Minimal | Patchy | Complete | Irreversible |
| | | loss | alopecia | alopecia, | alopecia |
| | | | | reversible | |
| Infection | None | Minor | Moderate | Major | Major with |
| (specify | | | | | hypotension |
| site) | | | | | |
| Heart | | | | | |
| Rhythm | Sinus | Sinus | Unifocal | Multifocal | Ventricular |
| | | tachycardia | PBC or | PVC | tachycardia |
| | | at rest | atrial | | |
| | | arrythmia | | | |
| Function | Noremal | No | Transient | Dysfunction | Dysfunction |
| | | symptoms | dysfunction | with | with |
| | | but | with | symptoms, | symptoms, |
| | | abnormal | symptoms, | requiring | no |
| | | cardiac sign | no therapy | therapy | responding |
| | | | required | | to therapy |
| Pericarditis | None | Effusion, no | Symptoms, | Tamponade, | Tamponade, |
| | | symptoms | no tap | tap required | surgery |
| | | | required | | required |
| Neurologic | | | | | |
| Conscious | Alert | Transient | Somnolent< | Somnolent> | Com |
| | | lethargy | 50% of | 50% of | |
| | | | waking | waking | |
| | | | hours | hours | |
| Peripheral | no | Parestheias, | Severe | Intolerable | Paralysis |
| nerves | symptoms | diminished | paresthesias | paresthesias | |
| | | tendon | mild | marked | |
| | | reflexes | weakness | motor loss | |
| Consti- | None | Mild | Moderate | Abdominal | Distention |
| pation (non- | | | | distention | and |
| narcotic) | | | | | vomiting |
| Pain | None | Mild | Moderate | Severe | Intractable |
| (treatment | | | | | |
| required) | | | | | |

### ADDENDUM 11

Total Protein Assay Procedure

CATALOGUE NUMBER 200-55 SIZE 4×125 mL

For the quantitative determination of total protein in serum. For IN VITRO diagnostic use.

### PRECAUTIONS

Avoid ingestion and contact with skin and eyes. See Material Safety Data Sheet. Do not pipette the reagent by mouth.

### REAGENTS

A solution containing 31.8 mmol/L sodium potassium tartrate, 12.0 mmol/L copper sulfate pentahydrate, 30.1 mmol/L potassium iodide, and 0.20 mol/L sodium hydroxide.

HISTORY

The use of the Biuret reaction as a method for the estimation of protein in plasma was first introduced by Reigler (1). Bomall, et. al. (2) modified the procedure by adding sodium potassium tartrate which acts as a complexing agent to form a stable cooper protein complex. This method uses the biuret reaction in which protein reacts with the reagent at an alkaline pH to form a blue-violet colored complex.

PRINCIPLE

At an alkaline pH the protein reacts with the copper in the Biuret reagent causing an increase in absorbance.

Protein +Cu⁺⁺>blue-violet complex

The increase in absorbance at 540 nm due to the formation of the colored complex is directly proportional to the concentration of protein in the reaction.

REAGENT PREPARATION

The reagent is provided in a ready to use form.

REAGENT STABILITY AND STORAGE

The reagents are stable at 18-26° C. until the expiry date stated on the labels.

REAGENT DETERIORATION

The reagent solution should be clear. Turbidity would indicate deterioration.

INSTRUMENTS

Any instrument with temperature control of ±0.5° C. that is capable of reading absorbance accurately with a sensitivity of 0.001 absorbance at 540 nm may be used. The bandwidth should be 10 nm or less, stray light 0.5% or less, and the wavelength accuracy within 2 nm.

SPECIMEN COLLECTION AND PREPARATION

Fresh, clear, unhemolysed serum is the specimen of choice.

INTERFERING SUBSTANCES

Hemolysed specimens should be avoided as hemoglobin falsely elevates total protein values.

PROCEDURE

Materials Provided

The reagent necessary for the determination of total protein is provided.

Materials required

1. An instrument which meets the requirements stated in the Instruments Section.

2. 1 cm cuvettes or a flow cell capable of transmitting light at 540 nm.

3. Test tubes of the appropriate size.

4. Pipettes of the appropriate size.

5. Deionized water.

6. An appropriate timer.

7. A protein standard for calibrating the procedure.

Conditions

Wavelength . . . 540 nm

Temperature . . . 18-26° C.

Pathlength . . . 1 cm

Mode . . . Endpoint

Reaction Time . . . 10 minutes

Sample Volume . . . 50 mL

Reagent Volume . . . 2.5 mL

Total Volume . . . 2.55 mL

Sample to Reagent Ratio . . . 50

Procedure

1. Into separate test tubes, pipette 50 mL of deionized water, protein standard, or serum to be assayed.

2. Add 2.5 mL of Total Protein Biuret Reagent and mix.

3. Incubate at 18-26° C. for 10 minutes.

4. Determine the absorbance of the standard (As) and of each unknown (A) at 540 nm using the deionized water sample as the reagent blank.

STABILITY OF FINAL REACTION MIXTURE

The color of the final reaction mixture is stable for one hour.

CALIBRATION

A protein standard is not included, however one should be used to calibrate the procedure.

QUALITY CONTROL

A normal and abnormal level control serum should be analyzed with each run of samples and the results should fall within plus or minus two standard deviations of the established value.

CALCULATION AND RESULTS

Results

Total protein concentration is expressed as g/L (g/dL).

Calculation

Total protein g/L (g/dL)=A×concentration of the standard

As

A=absorbance of the unknown

As=absorbance of the standard

Limitations

A sample with a total protein level exceeding the linearity limit should be diluted with 0.9% saline and reassayed incorporating the dilution factor in the calculation of the value. Severely lipemic or icteric samples require the use of sample blank which may be prepared using 50 mL of sample and 2.5 mL of distilled water.

EXPECTED VALUES (4)

These values are suggested guidelines. It is recommended that each laboratory establish the normal range for the area in which it is located.

PERFORMANCE CHARACTERISTICS

These performance characteristics were generated in DCL laboratories using automated procedures unless otherwise stated.

Recovery Study

Protein was added to 3 pools of sera to increase the total protein concentration by 25, 35, and 45 g/L. Recovery of the added protein averaged 96%.

Linear Range (NCCLS EP6-P)

This procedure is linear to 100 g/L.

Precision Studies (NCCLS EP5-T2)

Day to day precision was established by assaying two control sera twice a day for 10 days.

Total Protein Mean g/L Standard Deviation g/L Coefficient of Variation %

| | | | |
|---|---|---|---|
| Serum 1 | 48 | 0.8 | 1.7 |
| Serum 2 | 63 | 1.0 | 1.6 |

Within day precision was established by assaying two control sera twice a day for 10 days.

Total Protein Mean g/L Standard Deviation g/L Coefficient of Variation %

| | | | |
|---|---|---|---|
| Serum 1 | 48 | 0.3 | 0.6 |
| Serum 2 | 63 | 0.8 | 1. |

Accuracy (NCCLS EP9-P)

In an evaluation of this method a similar Biuret procedure was used as the reference method. Linear regression analysis gave the following equation.

This Method=1.037(reference method)−3.6 g/L.

### ADDENDUM 12

Procedure for Culture Sterility

1) MATERIAL REQUIRED: Culture loop calibrated, prepared blood agar plates, incubator.

2) Principle: Bacterial growth is checked by placing final product onto culture media. A calibrated loop is used to determine number of Colony Forming Units (CFU's)/mL.

3) Procedure: In the final preparation process, a calibrated loop of serum/tumor vaccine is taken to assess product sterility.

A) Take calibrated disposable loop and immerse in final (after second filtration) apply to culture plate in traditional streaking fashion.

B) Incubate for 12-24 hours at 36-38C. Check for colony growth.

C) Record results in logbook.

4) Summary: Double sterile filtration through a 0.2 μm filter should remove all viable organisms from the vaccine final product. A final check of the product will assure that the filtration system is functioning properly.

### ADDENDUM 13

LAL PROCEDURE

Limulus Amebocyte Lysate:

1) Intended use:

To determine the amount of endotoxin present in a solution. Detects endotoxin chromogenically. Used to establish endotoxin limits for pharmaceutical and medical devices, as an end-product endotoxin test, and developing a routine testing protocol.

2) Explanation of test:

Is a quantitative test for gram-negative bacterial endotoxin. A sample is mixed with the LAL supplied and insulated at 37 for ten minutes. Add a substrate solution and incubate for an additional six minutes at 37C. The reaction is then stopped. A yellow color is produced, and can be read spectrophotometrically at 405-410 nm. The value is then calculated from a standard curve.

3) Principle:

Gram-negative bacterial endotoxin catalyzes the activation of a proenzyme. The rate of activation is determined by the amount of endotoxin present. The splitting of p-nitroaniline (pNA) from the colorless substrate Ac-lle-Glu-Ala-Arg-pNA is done by catalyzing the activated enzyme. The pNA is measured at 405-410 nm photometrically, after the reaction is stopped. The absorbance and the endotoxin concentration is linear in the 0.1-1.0 EU/ml range. The values are calculated from the absorbance values of solutions containing known amounts of endotoxin standard.

4) Reagents:

A) LAL Reagent Water—Store at 2-8C

Two 30 ml bottles. Used to reconstitute all reagents and as a negative control (blank).

B) E. Coli Endotoxin—Store at 2-8C

One vial containing 15-30 EU lyophilized endotoxin. Reconstitute by adding 1.0 ml of LAL water warmed to room temperature. The value is predetermined and stated on an enclosed certificate of analysis. I.E. The value is 24 EU; mixed with 1.0 ml of water the concentration is them 24 EU/ml. Vortex for 15 minutes. Stable for one month at 2-8C. Must be warmed to room temperature prior to use and vortexed for 15 minutes. Important because endotoxin can attach to the glass.

C) Chromogenic substrate - Store at 2-8C

Two 7 mg vials of lyophilized substrate. Reconstitute by adding 6.5 ml of LAL Reagent Water, should yield a concentration of 2 mM. Stable for one month at 2-8C, if not contaminated with microorganisms or pyrogens.

D) Limulus Amebocyte Lysate (LAL) Store at 2-8C

Five vials containing lyophilized lysate, which is prepared from the circulating amebocytes of the horseshoe crab Limulus polyphemus. Reconstitute immediately before use with 1.4 ml of LAL Reagent Water. Swirl gently to avoid foaming. Reconstituted it can be stored at −10C or colder for up to one week if frozen immediately after reconstitution. Thaw and use only once. Protect from light.

5) Precautions and warnings:

A) All materials must be pyrogen-free.

B) Careful technique must be used to avoid microbial or pyrogen contamination.

C) Must abide to the times and temperature throughout the testing procedure.

D) If the container or diluent used to reconstitute the LAL has been entered previously or was not supplied by Biowhittaker, Inc., the diluent alone must be tested by endotoxin concentration.

Warnings: Laboratory use only. Not to determine endotoxin in man. For in vitro use.

6) Specimen Collection and Preparation:

All materials coming in contact with specimen or test reagents must be pyrogen-free. Take precautions to protect materials from environmental contamination.

Must keep pH of samples within the range of 7.4-8.0 by using pyrogen-free sodium hydroxide or hydrochloric acid. Measure the pH of the sample to avoid contamination by the pH electrode. Do not adjust unbuffered solutions.

Some compounds may give false positive or negative results, i.e. blood products, polynucleotides, solutions containing heavy metals or surfactants, or high ionic strength or osmolarity.

Store samples at 2-8C for less than 24 hours, more than 24 hours samples should be frozen.

7) Reagents required but not supplied:

Stop reagent (Acetic acid, 25% v/v glacial acetic acid in water) Sodium hydroxide, 0.1 N, dissolved in LAL Reagent Water. Hydrochloric acid, 0.1 N, diluted in LAL Reagent Water.

8) Materials required by not supplied:

Disposable pyrogen-free glass dilution tubes. Individually wrapped measuring pipettes. Automatic hand-held pipettes with sterile, individually wrapped tips.

Disposable pyrogen-free microplates

Optional: 8 channel pipettor

Reagent reservoirs

9) Equipment required but not supplied:

Dry Bath/multi-Block heater at 37C+1.0C

Stop watch

Vortex mixer

Microplate reader

Tube block for heater

Microplate adaptor for heater

10) Preliminary reagent preparation:

For standard endotoxin solutions should be used, made up from the endotoxin supplied in the hit.

A) Prepare a solution containing 1.0 EU/ml of the endotoxin by diluting 0.1 ml of the endotoxin stock solution with (x-1)/10 ml of LAL Reagent Water. X equals the endotoxin concentration. Vortex 1 min.

Example: If x=23 EU/ml than:

(23-1)/10, LAL Reagent Water

dilute 0. 1 ml of the endotoxin with 2.2 ml of LAL Reagent Water

B) Transfer 0.5 ml of this 1.0 EU/ml solution into 0.5 ml of LAL Reagent Water.

Label 0.5 EU/ml. Vortex 1 min.

C) Transfer 0.5 of the 1.0 EU/ml solution into 1.5 ml of LAL Reagent water. Label 0.25 EU/ml. Vortex 1 min.

D) Transfer 0.1 ml of the 1.0 EU/ml solution into 0.9 of LAL Reagent water. Label 0.1 EU/ml. Vortex 1 min.

11) Test procedure:

The addition of all reagents must be consistent. All microplate wells must be treated exactly the same in order to determine proper endotoxin concentration. Pipette in the same order from row to row, at the same rate.

A) Pre-heat the microplate at 37C+1.0C in the heating block adaptor.

B) Dispense 50 μl of sample or standard into the appropriate microplate well. Including one blank, four standards, then the patient's.

C) At T=0 add 50 UL of LAL, use a multichannel pipettor, begin time when LAL is added to first row. Be consistent with rate of pipetting. After LAL has been added to all wells, remove the plate from the incubator and gently tap sides to mix. Return plate to heating block and replace cover.

D) At T=10 minutes, add 100 UL of substrate solution prewarmed at 37C +1.0C. Maintain consistent pipetting rate. When substrate is added to all wells remove plate again, gently tap for mixing, and return to heat block, replace cover.

E) At T=16 minutes, add 100 UL of stop reagent. Maintain consistent pipetting rate. After adding remove plate and repeatedly tap the side to mix.

F) After mixing, place on multi-well reader and read at 405-410 nm.

12) Calculation of Endotoxin Concentration;

The absorbance at 405-410 nm is linear in the concentration range of 0.1 to 1.0 EU/ml endotoxin.

Calculator Method:

A calculator equipped with linear regression capability can be used. Enter the mean delta absorbance and concentration of the four standards. Determine the corresponding endotoxin concentration of the samples from their absorbance by linear regression.

*Note:

If the concentration of endotoxin in the sample is greater than 1.0 EU/ml, make a 5-fold dilution using LAL Reagent Water and retest. Calculate the concentration of the diluted sample and multiply by 5.

Performance Characteristics:

Linearity:

The linearity of the standard curve within the concentration range used to predict endotoxin values should be verified. No less than 4 endotoxins standards, spannings from 0.1 to 1.0 EU/ml range, should be assayed along with a blank.

The coefficient of correlation, r, for the individual mean delta absorbance of the standards (at least 16 points) vs. their respective endotoxin concentration should be >0.980.

13) Product Inhibition:

When substances in the sample interfere with the LAL reaction, this is product inhibition. This inhibition can cause lower levels of endotoxin than may actually be present in the test sample. The lack of product inhibition should be determined, for either undiluted or at an appropriate dilution.

To verify the lack of product inhibition a spike of the sample is made using a known amount of endotoxin. The spike solution is assayed alongside the unspiked solution. Then the respective endotoxin concentrations are determined. The difference of the endotoxin values should equal known concentration of the spike +25%.

To prepare the spike aliquots:

1) Prepare a 1.0 EU/ml endotoxin solution in test sample by diluting the endotoxin stock solution 1/x, where x is the endotoxin concentration of stock in EU/ml. Use the test sample as the diluent. Vortex for 1 minute.

2) To prepare a 0.4 EU/ml endotoxin solution in test sample, dilute the 1.0 EU/ml solution ½.5 using the test sample as the diluent. Combine 1.0 ml of the 1.0 EU/ml solution in test sample with 1.5 ml of test sample. Vortex for 1 minute.

If the sample is found inhibitory to the LAL reaction, further diluting may be necessary, until the inhibition is overcome. Initially, one may want to screen for inhibition by testing 10-fold dilutions of test samples. When the approximate non-inhibitory dilution is determined, the exact dilution can be found by testing two-fold dilutions around the dilution.

Limitations:

The degree of inhibition or enhancement is dependent upon the concentration of product. It is necessary to establish performance characteristics for each independently, if several concentrations of the same product are to be assayed.

It may be necessary to adjust the pH of the sample to overcome inhibition.

14) Colored Samples:

Certain samples may possess significant color, also using 25% acetic acid as the stop reagent, may cause samples to turn yellow, such as certain tissue culture media.

A quick mock reaction tube test can be performed to check for color change. Add 50 μL of sample, 150 μL H₂O and 100 μL appropriate stop reagent with incubation. Read the absorbance of this solution at 405-410 nm. If absorbance is significantly greater than the LAL Reagent Water, the color of the product must be taken into account.

When ready to assay, prepare a sample blank by confirming 50 UL sample, 150 μL H₂O and 100 μL appropriate stop reagent with incubation. Assay along with the appropriate standards and LAL Reagent Water blank. Calculate the delta absorbance by subtracting the absorbance of the LAL Reagent Water blank. Use only the LAL Reagent Water blank to calculate delta absorbance for the endotoxin standards and non-colored products.

If concentration of the color sample is >0.5 absorbance units, the sample should be diluted and reassayed. The dilution factor is then used in the final calculations for determining the concentration of endotoxin.

15) References:

A) Bang F. B. A. A bacterial disease of Limulus polyphemus. Bull. Johns Hopkins Hosp. 98:325 (1956).

B) Levin J. and F. B. Bang. The role of endotoxin in the extracellular coagulation of Limulus blood. Bull. Johns Hopkins Hosp. 115:265 (1964).

C) Levin J. and F. B. Bang. A description of cellular coagulation in the Limulus. Bull. Johns Hopkins Hosp. 115:337 (1964).

D) Levin J. and F. B. Bang. Clottable protein in Limulus: its localization and kinetics of its coagulation by endotoxin. Thromb. Diath. Haemouh. 19:186 (1968).

F) LAL Package insert, 1997.

### ADDENDUM 14

TNF Procedure

MATERIALS

1. Materials Provided:

Anti-Human TNF Precoated Strip Well Plate—1 (96 wells)

Lyophilized yeast-derived Recombinant Human TNF Standard—2 vials

Standard Diluent—1 vial, (12 ml), contains 0.01% thimerosal

Biotinylated Antibody Reagent—1 vial, (8 ml), contains 0.01% thimerosal

30X Wash Buffer—1 vial, (50 ml)

Streptavidin-HRP Concentrate—1 vial, (0.075 ml)

Streptavidin-HRP Dilution Buffer—1 vial, (14 ml), contains 0.01% thimerosal

Premixed TMB Substrate Solution—1 vial, (13 ml)

Stop Solution—1 vial, (14 ml)

Adhesive Plate Covers, 4

Instruction Booklet, 1

Data Templates, 2

Recyclable Box and Insert, 1 each

2. Additional Materials Required:

Precision pipettors with disposable plastic tips to deliver 5 to 1000 μl.

Plastic pipettes to deliver 5 to 15 ml.

Distilled or deionized water for Wash Buffer and standard reconstitution.

A glass or plastic 2 liter container to prepare Wash Buffer.

A squirt wash bottle, or an automated immunoplate washer.

1.5 ml polypropylene or polyethylene tubes to prepare standards. Do not use polystyrene, polycarbonate or glass tubes.

4 disposable reagent reservoirs.

15 ml plastic tube to prepare Streptavidin-HRP Solution.

A microcentrifuge for preparing Streptavidin-HRP Solution.

A standard ELISA reader for measuring absorbance at 450 nm and 550 nm. If a 550 nm filter is not available, the absorbance can be read at 450 nm only. Refer to the instruction manual supplied with your instruction manual supplied with your instrument.

Graph paper or a computerized curve-fitting statistical software package.

PRECAUTIONS

a) All specimens and reagents must be at room temperature (20-25° C.) before use in the ELISA.

b) Vigorous washing of the plate is essential.

c) You may wish to validate your media when running culture supernatants with serum, urine or plasma in the same plate. Prepare a standard curve (including a zero/blank) using your culture medium. This curve should be run in parallel with a standard curve prepared with culture medium. This curve should be run in parallel with a standard curve prepared with Standard Diluent. If the OD values of the two curves are within 10% then the assay can be run using a standard curve prepared with Standard Diluent, whether you are testing culture supernatant, plasma or serum samples.

d) When preparing standard curve and sample dilution in your culture medium, use the same medium you used to culture the cells. For example, if RPMI with 10% fetal calf serum (FCS) was used to culture the cells, then RPMI with 10% FCS should be used to dilute the standard and samples. Do NOT use RPMI without serum supplement.

e) Do not use 37° or 56° water baths to thaw samples. Thaw samples at room temperature.

f) If using a multichannel pipettor, always use a new disposable reagent reservoir for the addition of Biotinylated Antibody Reagent, Streptavidin-HRP Solution, TMB Substrate Solution and Stop Solution.

g) Use fresh disposable pipette tips for each transfer to avoid cross contamination.

h) Use a new adhesive plate cover for each incubation step in the ELISA.

i) Do not mix reagents from different kit lots.

j) Avoid microbial contamination of reagents.

k) Avoid exposure of reagents to excessive heat or light during storage and incubation.

l) If using samples that are clotted, grossly hemolyzed or microbially contaminated, or if there is any question about the integrity of a sample, make a note on the template and interpret results with caution.

m) Individual components of this assay kit contain preservatives and antibiotics. Gloves should be worn while performing the assay to avoid contact with samples and reagents.

n) Do not use glass pipettes to measure out the TMB Substrate Solution.

o) Care must be taken not to contaminate the TMB Substrate Solution. If the solution is blue prior to use, DO NOT USE.

SAMPLE PREPARATION

1. Handling and Storage:

Serum; EDTA, sodium citrate and heparin plasma; and culture supernatant may be tested in the ELISA.

50 ul of sample per well is required in this assay.

Samples that are to be assayed within 12 hours should be stored at 2-8° C. When storing samples for longer periods of time aliquot and freeze them at −70° C.

Test samples should be assayed in duplicate.

Avoid freezing and thawing samples more than once.

Bring samples gently to room temperature before running the assay. Mix samples by gently inverting the tubes. Do not use 37° C. or 56° water baths to thaw samples.

2. Sample Dilution:

If you suspect the cytokine concentration of a sample exceeds the highest point of the standard curve, 1000 pg/ml, we suggest preparing one or more ten-fold dilutions of the test sample. When testing culture supernatants prepare the serial dilutions using your culture medium. When testing serum or plasma prepare the serial dilutions using the Standard Diluent provided. For example, a ten-fold dilution is prepared by adding 0.05 ml (50 ul) of test sample to 0.45 ml (450 ul) of appropriate diluent. Mix thoroughly between dilutions before assaying.

REAGENT PREPARATION

1. WashBuffer:

Label a clean glass or plastic 2 liter container “Wash Buffer.” 30X Wash Buffer may have a cloudy appearance. Add the entire contents of the 30X Wash Buffer bottle and dilute to a final volume of 1.5 liters with distilled or deionized water. Mix thoroughly. Wash Buffer should be a room temperature prior to use in the assay. If running partial plates, keep the reconstituted Wash Buffer at 2-8° C.

2. Streptavidin-HRP Solution

a) Prepare Streptavidin-HRP Solution no more than 5 minutes prior to use. Do not prepare more than required. See step e below when running partial plates.

b) Do not store prepared Streptavidin-HRP Solution.

c) Use a 15 ml plastic tube to prepare the Streptavidin-HRP Solution. You may wish to briefly microcentrifuge the Streptavidin-HRP Concentrate to force the entire contents to the bottom of the tube.

d) Add 30 μl (“ul”) of Streptavidin-HRP Concentrate to 12 ml Streptavidin-HRP Dilution Buffer and mix gently. This will give you the Streptavidin-HRP Solution.

e) Use only what is required for the number of strips you are running. Use 2.5 μl (“ul”) of Streptavidin-HRP Concentrate and 1 ml of Streptavidin-HRP Dilution Buffer for each strip being run.

3. Standards:

a) Two vials of lyophilized standards are provided with this kit. Reconstitute and use one vial per partial plate.

b) Prepare Standards shortly before use. Use within one hour of reconstitution. Do not store reconstituted standards.

c) When running culture supernatant samples, reconstitute standard in distilled or deionized water. Reconstitution volume is stated on the standard vial label. The standard will take approximately 1 minute to dissolve. Mix by gently inverting the vial. Use your culture medium to prepare the dilutions of the Standard Curve, go to step e below for further instructions. See the Precautions section step c for media validation procedure. If running a partial plate, refer to step a above.

d) If running serum or plasma samples, reconstitute standard with distilled or deionized water. Reconstitution volume is stated on the standard vial label. The standard will take approximately 1 minute to dissolve. Mix by gently inverting the vial. Use the Standard Diluent provided to prepare the dilutions of the Standard Curve. If running a partial plate, refer to step a above.

e) Label 6 tubes, one for each standard curve point: 1000 pg/ml, 400 pg/ml, 160 pg/ml, 64 pg/ml, 25.6 pg/ml and 0 pg/ml. Then prepare 1:2.5 serial dilutions for the standard curve as follows:

f) Pipette 240 ul of appropriate diluent (see steps c and d above) into each tube.

g) Pipette 160 ul of the reconstituted standard into the first tube labeled 1000 pg/ml and mix.

h) Pipette 160 ul of this dilution into the second tube labeled 400 pg/ml and mix.

Repeat serial dilutions three more times. These concentrations, 1000, 400, 160, 64, 25.6 and 0 pg/ml are your standard curve.

ASSAY PROCEDURE

1 . Before You Start:

a) Review the entire contents of this instruction booklet.

b) Verify all components in the ELISA kit against the Materials Provided list on page 2.

c) Be sure to assemble all Additional Materials Required listed on page 2 prior to running this assay.

2. Sample and Biotinylated Antibody Reagent Incubation:

a) Determine the number of strips you wish to run. Leave these strips in the plate frame. Place the remaining unused strips back in the foil pouch with the desiccant provided. Store these reserved strips at 2-8° C., making sure the foil pouch is sealed tightly. After running assay, retain plate frame for second partial plate. When running the second partial plate, place the reserved strips securely in the plate frame.

b) Use the Data Template provided to record the locations of the zero standard, hTNF Standards and test samples. Five Standards and one zero standard should be run in duplicate with each series of unknown samples.

c) Add 50 ul of reconstituted standards or test samples in duplicate to each well. If you suspect that the TNFa concentration in any test sample exceeds the highest point on the standard curve, 1000 pg/ml, see Sample Preparation step 2. If you are not adding samples or standards to any wells being utilized add 50 ul of Standard Diluent to these empty wells.

d) If using a multichannel pipettor, use a new reservoir to add the Biotinylated Antibody Reagent. Remove from the vial only the amount required for the number of strips being used.

e) Add 50 ul of the Biotinylated Antibody Reagent to all wells that will be used. Be careful not to touch samples in the wells with the pipette tip to avoid cross-contamination.

f) Carefully cover the plate with an adhesive plate cover, making sure all edges and strips are sealed tightly. Do this by running your thumb over the edges and down each strip. Incubate for two (2) hours at room temperature, 20-25° C.

g) At the end of the incubation period, carefully remove the adhesive plate cover. Wash the plate THREE times with Wash Buffer, using the procedure described below.

3. Wash the Plate:

Gently squeeze the sides of the plate frame when washing the plate to assure that all strips remain securely in the frame.

Manual Wash: Decant the contents of the plate into a sink or other receptacle. Using a squirt bottle, vigorously fill each well completely with Wash Buffer, then decant the total of THREE washes. Pat onto paper towels or other absorbent material.

Automated: Aspirate all wells and wash THREE times with Wash Buffer. Repeat the procedure two more times for a total of THREE washes. Pat onto paper towels or other absorbent material.

4. Streptavidin-HRP Solution Incubation:

a) Using a single or multichannel pipettor, add 100 ul of prepared Streptavidin-HRP Solution, see Reagent Preparation step 2, to each well. If using a multichannel pipettor use a new reagent reservoir when adding the prepared Streptavidin-HRP Solution.

b) Carefully attach a new adhesive plate cover, making sure all edges and strips are sealed tightly. Incubate the plate for 30 minutes at room temperature, 20-25° C.

c) At the end of the Streptavidin-HRP Solution incubation, carefully remove the adhesive plate cover and wash THREE times using the procedure described below.

5. Wash the Plate:

Gently squeeze the sides of the plate frame when washing the plate to assure that all strips remain securely in the frame.

Manual Wash: Decant the contents of the plate into a sink or other receptacle. Using a squirt bottle, vigorously fill each well completely with Wash Buffer, then decant the total of THREE washes. Pat onto paper towels or other absorbent material.

Automated: Aspirate all wells and wash THREE times with Wash Buffer. Repeat the procedure two more times for a total of THREE washes. Pat onto paper towels or other absorbent material.

6. Substrate Incubation and Stop Step:

a) Use different disposable reagent reservoirs when adding the TMB Substrate Solution.

b) Dispense from the bottle ONLY the amount required for the number of strips you are running, 100 ul per well. Do not use a glass pipet to measure out the TMB Substrate Solution. Do not combine leftover substrate with the reserved for the second half plate. Care must be taken to ensure that the remaining TMB Substrate Solution is not contaminated.

c) Pipette 100 ul of TMB Substrate Solution into each well.

d) Allow enzymatic color reaction to develop at room temperature for 30 minutes. THE PLATE SHOULD BE DEVELOPED IN THE DARK. Do not cover the plate with aluminum foil or a plate sealer. The substrate reaction yields a blue color than turns yellow when Stop Solution is added.

e) After 30 minutes, stop the reaction with the Stop Solution provided, adding 100 ul to each well.

7. Read the Absorbance:

Measure the absorbance on an ELISA reader set at 450 and 550 nm. Subtract readings of 550 mn from the readings at 450 nm. Reading at dual wavelengths will correct for optical imperfections in the microtiter plate. If a wavelength correction is not available, read the plate at 450 nm. Readings at 450 nm only can be used, but absorbances may be higher. THE PLATE MUST BE READ WITHIN 30 MINUTES OF STOPPING THE REACTION.

CALCULATION OF RESULTS

a) The standard curve is used to determine the amount of TNF is an unknown sample. The standard curve is generated by plotting the average absorbance (450-550 nm) obtained for each of the Standard concentrations on the vertical (Y) axis versus the corresponding TNF concentration on the horizontal (X) axis.

b) Calculate your results manually using graph paper or with a curve-fitting statistical software package. If using a curve fitting software package plot a 4-parameter curve fit. The amount of TNF in each sample is determined by interpolating from the absorbance value (Y axis) to the TNF concentration (X axis) using the standard curve.

c) If a dilution was performed on a test sample, multiply the value interpolated from the standard curve by the dilution factor to calculate the pg/ml of TNF in the sample.

d) Optical Density values obtained for duplicates should be within 10% of the mean. Duplicate values that differ from the mean by greater than 10% should be considered suspect and should be repeated.

PERFORMANCE CHARACTERISTICS

1. Sensitivity:

<5 pg/ml

The sensitivity of this assay, or Lower Limit of Detection (LLD)1, was determined by assaying replicates of the zero and the standard curve. The mean signal of zero+2 standard deviations read in dose from the standard curve is the LLD. This value is the smallest dose that is not zero with 95% confidence.

2. Precision:

The intra-assay coefficient of variation is plotted against TNF concentration (pg/ml). The points represent samples run in replicates of four in six different kit lots.

3. Specificity:

This ELISA is specific for the measurement of natural and recombinant human TNF. It does not cross react with human; IL-1, IL-1, IL-2, IL-3, IL-4, IL-6, IL-7, IL-8, TNF, IFNγ; or mouse TNF.

14. Expected Values:

Human TNF was not detected in each of five normal serum samples. The average level of human TNF detect in five plasma samples was 1.4 pg/ml with a range of 0 to 4.9 pg/ml.

5. Recovery:

Recovery in this ELISA was determined by spiking natural human TNF into normal human serum and plasma samples, collected from apparently healthy individuals, as well as a control buffer. Mean recoveries were as follows:

| | | | |
|---|---|---|---|
| Control Dose | 696 pg/ml | 362 pg/ml | 132 pg/ml |
| Mean Serum Recovery | 122% +/− 7% | 125% +/− 6% | 88% +/− 4% |
| (+/− SD) | | | |

| | | | |
|---|---|---|---|
| Control Dose | 929 pg/ml | 393 pg/ml | 296 pg/ml |
| Mean Plasma | 124% +/− 7% | 124 +/− 7% | 90% +/− 7% |
| Recovery (+/− SD) | | | |

6. Linearity of Dilution:

Linearity of Dilution was determined by serially diluting nine different positive samples. The dilutions were run in the ELISA and the “observed” doses are plotted against the “expected” doses.

ASSAY PARAMETERS

1. Assay Range

0-1000pg/ml

Suggested standard curve points are 1000, 400, 160, 64, 25.6 and 0 pg/ml. If you choose to prepare a different series of standard curve points, do NOT exceed the highest point of the standard curve, 1000 pg/ml.

2. Calibration:

The standards in this ELISA are calibrated to WHO reference lot 87/650. One (1) pg of

Endogen Standard=1 WHO pg=0.04 WHO units.

3. Reproducibility:

Intra-Assay CV: <10%

Inter-Assay CV: <10%

STABILITY AND STORAGE

a) Refer to the expiration date stamped on the kit box.

b) Store all reagents at 2-8C upon receiving. Do not freeze reagents.

c) Do NOT use kit beyond stated expiration date.

### ADDENDUM 15

Mycoplasma (PPLO) Culture Procedure

PURPOSE

To provide evidence that the final vaccine product is PPLO (Mycoplasma) free.

PROCEDURE

Mycoplasmas may be grown in enriched medium containing heart infusion agar, horse serum, yeast extract, and penicillin G.

1) Plate a loopful of final product on selective media and streak for isolation.

2) Growth of colonies is observed microscopically under 40X magnification and appear as large (250 to 750 ul) to small (1 to 10 ul) raised, pitted, and lacy to coarsely pebbled colonies. The central part of the colony grows into the agar medium appearing darker than the periphery when examined by transmitted light, giving it a characteristic “fried egg” appearance. Plates should be observed for colonies at 7, 14, and 21 days. Final observation and discard will occur at 30 days. Results shall be logged in vaccine logbook.

REFERENCE

Bailey and Scott, Diagnostic Microbiology

### ADDENDUM 16

IFN Procedure

MATERIALS

1. MATERIALS NEEDED

Anti-Human IFNy Precoated Strip Well Plate—1 (96 well)

Lyophilized E. coli-derived Recombinant Human IFNγ Standard—2 vials

Standard Diluent—1 vial (12 ml), contains 0.01% thimerosal

Biotinylated Antibody Reagent—1 vial (8 ml), contains 0.01% thimerosal

30X Wash Buffer—1 vial (50 ml)

Streptavidin-HRP Concentrate—1 vial (0.075 ml)

Streptavidin-HRP Dilution Buffer—1 vial (14 ml), contains 0.01% thimerosal

Premixed TMB Substrate Solution—1 vial (13 ml)

Stop Solution—1 vial (14 ml)

Adhesive Plate Covers—4

Instruction Booklet—1

Data Templates—2

Recyclable Plastic Box and Insert—1 each

2. ADDITIONAL MATERIALS REQUIRED

Precision pipettors with disposable plastic tips to deliver 5 to 1000 ul.

Plastic pipets to deliver 5 to 15 ml.

Distilled or deionized water for Wash buffer and standard reconstitution.

A glass or plastic 2 liter container to prepare Wash Buffer.

A squirt wash bottle, or an automated immunoplate washer. 1.5 ml polypropylene or polyethylene tubes to prepare standards. Do not use polystyrene, polyethylene tubes to prepare standards. Do not use polystyrene, polycarbonate or glass tubes.

4 disposable reagent reservoirs.

15 ml plastic tube to prepare Streptavidin-HRP solution.

A microcentrifuge for preparing Streptavidin-HRP solution.

A standard ELISA reader for measuring absorbance at 150 nm and 550 nm. If a 550 nm filter is not available, the absorbance can be read at 450 nm only. Refer to the instruction manual supplied with your instrument.

Graph paper or a computerized curve-fitting statistical software package.

3. PRECAUTIONS

a) Vigorous washing of the plate is essential.

b) You may wish to validate your media when running culture supernatant with serum, urine or plasma in the same plate. Prepare a standard curve (including a zero/blank) using your culture medium. This curve should be run in parallel with 10% of the mean for both curves, then the assay can be run using a standard curve prepared with Standard Diluent, whether you are testing culture supernatant, plasma, urine or serum samples.

c) All specimens and reagents must be a room temperature (20-25C) before use in the ELISA.

d) Do not use water baths to thaw samples or reagents. Thaw samples and reagents at room temperature.

e) If using a multi-channel pipettor, always use a new disposable reagent reservoir for the addition of Biotinylated Antibody Reagent, Streptavidin-HRP solution, TMB Substrate Solution and Stop Solution.

f) Use fresh disposable pipette tips for each transfer to avoid cross contamination.

g) Use a new adhesive plate cover for each incubation step in the ELISA.

h) Do not mix reagents from different kit lots.

i) Avoid microbial contamination of reagents.

j) Avoid exposure of reagents to excessive heat or light during storage and incubation.

k) If using samples that are clotted, grossly hemolyzed or microbially contaminated or if there is any question about the integrity of a sample, make a note on the template and interpret results with caution.

l) Individual components of this assay kit contain antibiotics and preservatives. Gloves should be worn while performing the assay to avoid contact with samples and reagents. Please follow proper disposal procedures.

m) Do not use glass pipettes to measure out the TMB Substrate Solution.

n) Care must be taken not to contaminate the TMB Substrate Solution. If the solution is blue prior to use, DO NOT USE.

4. SAMPLE PREPARATION

1. Handling and Storage:

Serum; EDTA, sodium citrate and heprin plasma; urine and culture supernatants mat be tested in the ELISA

50 μl (“ul”) of sample per well is required per well in this assay. Samples that are to be assayed within 24 hours should be stored at 2-8C. When storing samples for longer periods of time aliquot and freeze than at −70C.

Test samples should be assayed in duplicate each time the ELISA is performed. Avoid freezing and thawing samples more than once.

Bring samples gently to room temperature before running the assay. Mix samples by gently inverting the tubes. Do not use 37C or 56C water baths to thaw samples.

2. Sample Dilution:

If you suspect the cytokine concentration of sample exceeds the highest point of the standard curve, 1000 pg/ml, we suggest preparing one or more ten-fold dilutions of the test sample. When testing culture supernatants prepare the dilutions using your medium. When testing serum, plasma, or urine prepare the dilutions using the Standard Diluent provided. For example, a ten-fold dilution is prepared by adding 0.05 ml (50 ul) of test sample to 0.45 ml (450 ul) of appropriate diluent. Mix thoroughly between dilutions before assaying.

5. REAGENT PREPARATION

1. Wash Buffer:

Label a clean glass or plastic 2 liter container “Wash Buffer”. 30X wash buffer may have cloudy appearance. Add the entire contents of the 30X Wash Buffer bottle and dilute to a final volume of 1.5 liters with distilled or deionized water. Mix thoroughly. Wash Buffer should be at room temperature prior to use in the assay. If running partial plates, store the reconstituted Wash Buffer at 2.8 C. Do not use Wash Buffer if it becomes visibly contaminated during storage.

2. Streptavidin-HRP Solution:

a) Prepare Streptavidin-HRP Solution no more than 15 minutes prior to use. Do not prepare more than required. See step f below when running partial plates.

b) Do not store prepared Streptavidin-HRP Solution.

c) Use a 15 ml plastic tube to prepare the Streptavidin-HRP Solution.

d) You may wish to microcentrifuge the Streptavidin-HRP Concentrate to force the entire contents of the vial to the bottom.

e) Add 30 ul of Streptavidin-HRP Concentrate to 12 ml of Streptavidin-HRP Dilution buffer and mix gently. This will give you the Streptavidin-HRP Solution. If running partial plates see step f.

f) Use only what is required for the number of strips being run. Use 2.5 ul of Streptavidin-HRP Concentrate and 1 ml of Streptavidin-HRP Diluted Buffer for each strip being run.

3. Standards:

a) Two vials of lyophilized standards are provided with this kit. Reconstitute and use on vial per partial plate.

b) Prepare Standards shortly before use. Use within one hour of reconstitution. Do not store reconstituted standards.

c) When running culture supernatant samples, reconstitute standard in distilled or deionized water. Reconstitution volume is stated on the standard vial label. The standard will take approximately 1 minute to dissolve. Mix by gently inverting the vial. Use your culture medium to prepare the dilutions of the Standard Curve, go to step e below for further instructions. See section E.b for media validation procedure.

d) If running serum, plasma, or urine samples, reconstitute standard with distilled or deionized water. Reconstitution volume is stated on the standard vial label. The standard will take approximately 1 minute to dissolve. Mix by gently inverting the vial. Use Standard Diluent provided to prepare the dilutions of the Standard Curve.

e) Label 6 tubes on for each standard curve point: 1000, 400, 160, 64, 25.6, and 0 pg/ml. Then prepare 1;2.5 serial dilutions for the standard curve as follows:

f) Pipette 240 ul of appropriate diluent (see steps c and d above) into each tube.

g) Pipette 160 ul of the reconstituted standard into the first tube labeled 1000 pg/ml and mix.

h) Pipette 160 ul of this dilution into the second tube labeled 400 pg/ml and mix.

i) Repeat the serial dilutions three more times. These concentrations, 1000, 400, 160, 64, 25.6, and 0 pg/ml are standard curve.

6. ASSAY PROCEDURE

1. Sample and Biotinylated Antibody Reagent Incubation:

a) Determine the number of strips you wish to run. Leave these strips in the plate frame. Place the remaining unused strips back in the foil pouch with the desiccant provided. Store these reserved strips at 2-8C, making sure the foul pouch is sealed tightly. After running the assay, retain the plate frame for the second partial plate. When running the second partial plate, place the reserved strips securely in the plate frame.

b) Use the Data Template provided to record the locations of the zero standard (blank or negative control), IFNy Standards and test samples. Five standards and one blank should be run in duplicate with each series of unknown samples.

c) If using a multichannel pipettor, use a new reservoir to add the Biotinylated Antibody Reagent. Remove from the vial only the amount required for the number of strips being used.

d) Add 50 μl (“ul”) of Biotinylated Antibody Reagent to all wells that will be used.

e) Add 50 ul of reconstituted standards or test samples in duplicate to each well. If you suspect that the IFNy concentration in any test sample exceeds the highest point on the standard curve, 1000 pg/ml, see section F.2. If you are not adding samples or standards to any wells being utilized add 50 ul of Standard Diluent to these empty wells.

f) Carefully cover the plate with an adhesive plate cover, making sure all edges and strips are sealed tightly. Do this by running your thumb over the edges and down each strip. Incubate for two (2) hours at room temperature 20-25C.

g) At the end of the incubation period, carefully remove the adhesive plate cover. Wash the plate THREE times with Wash Buffer, using the procedure described below.

2. Wash the Plate:

Gently squeeze the long sides of the plate frame when washing the plate to assure that all strips remain securely in the frame.

Manual Wash: Decant the contents of the plate into a sink or other receptacle. Using a squire bottle, vigorously fill each completely with Wash Buffer, then decant the contents into a sink or other receptacle. Repeat the procedure two or more times for a total of THREE washes. Pat onto paper towels or other absorbent material.

Automated: Aspirate all wells and fill each well completely with Wash Buffer. Repeat the procedure two or more times for a total of THREE washes. Pat onto paper towels or other absorbent material.

3. Streptavidin-HRP Solution Incubation:

a) Using a single or multichannel pipettor, add 100 ul of prepared Streptavidin-HRP Solution; see section G.2, to each well. If using a multichannel pipettor use a new reagent reservoir when adding the prepared Streptavidin-HRP Solution.

b) Carefully attach a new adhesive plate cover, making sure all edges and strips are sealed tightly. Incubate the plate for 30 minutes at room temperature, 20-25.

c) At the end of the Streptavidin-HRP Solution incubation, carefully remove the adhesive plate cover and wash THREE times using the procedure described below.

4. Wash the Plate:

Gently squeeze the long sides of the plate frame when washing the plate to assure that all strips remain securely in the frame.

Manual Wash: Decant the contents of the plate into a sink or other receptacle. Using a squire bottle, vigorously fill each completely with Wash Buffer, then decant the contents into a sink or other receptacle. Repeat the procedure two or more times for a total of THREE washes. Pat onto paper towels or other absorbent material.

Automated: Aspirate all wells and fill each well completely with Wash Buffer. Repeat the procedure two or more times for a total of THREE washes. Pat onto paper towels or other absorbent material.

5. Substrate Incubation and Stop Step:

a) Use different disposable reagent reservoirs when adding the TMB Substrate Solution and Stop Solution.

b) Dispense from the bottle ONLY the amount required for the number of strips you are running, 100 ul per well. Do not use a glass pipette to measure out the TMB Substrate Solution. Do not combine leftover substrate with that reserved for the second half plate. Care must be taken to ensure that the remaining TMB substrate is not contaminated.

c) Pipette 100 ul of TMB substrate Solution into each well.

d) Allow enzymatic color reaction to develop at room temperature for 30 minutes. THE PLATE SHOULD BE DEVELOPED IN THE DARK. Do not cover the plate with aluminum foil or a plate sealer. The substrate reaction yields a blue solution that turns yellow when Stop Solution is added.

6. Read the Absorbance:

Measure the absorbance on an ELISA reader set at 450 and 550 nm. Subtract readings of 550 nm from the readings at 450 nm. Reading at dual wavelengths with correct for optical imperfections in the micrometer plate. If a wavelength correction is not available read the plate at 450 nm. Reading at 450nm only can be used, but absorbances may be higher. THE PLATE MUST BE READ WITHIN 30 MINUTES OF STOPPING THE REACTION.

7. CALCULATION OF RESULTS

a) The standard curve is used to determine the amount of IFN-gamma in an unknown sample. The standard curve is generated by plotting the average absorbance (450-550 nm) obtained for each of the Standard concentrations on the vertical (Y) axis versus the corresponding IFN-gamma concentration on the horizontal (X) axis.

b) Calculate your results manually using graph paper or with a curve-fitting statistical software package. The amount of IFNγ in each sample is determined by interpolating from the absorbance value (Y axis) to the IFNγ concentration (X axis) using the standard curve.

c) If a dilution was performed on a test sample, multiply the value interpolated from the standard curve by the dilution factor to calculate the pg/ml of IFNγ in the sample.

d) Optical Density values obtained for duplicates should be within 10% of the mean. Duplicate values that differ from the mean by greater than 10% should be considered suspect and should be repeated.

8. PERFORMANCE CHARACTERISTICS

1. Sensitivity: <2 pg/ml

The sensitivity of this assay, or Lower Limit of Detection (LLD), was determined by assaying replicates of zero and the standard curve. The mean signal of zero +2 standard deviations read in dose form the standard curve is the LLD. This value is the smallest dose that is not zero with 95% confidence.

2. Assay Range: 0-1000 pg/ml

Suggested standard curve points are 1000, 400, 160, 64, 25.6, and 0 pg/ml.

3. Precision:

The intra-assay coefficient of variation is plotted against IFNy concentration (pg/ml). The points represent samples run in replicates of four in four different kit lots.

4. Reproducibility:

Intra-Assay CV: <10%

Intra-Assay CV: <10%

5. Specificity:

This ELISA is specific for the measurement of natural and recombinant human IFNγ. It does not cross react with human; IL-1, IL-1, IL-2, IL-3, IL-6, IL-7, IL-8, TNF or GM-CSF.

6. Calibration:

The standards in this ELISA are calibrated to the NIAID recombinant IFNγ standard lot Gg23-901-530. One (1) pg of Endogen standard=56 pg of NIAID standard=0.04 NIAID units.

7. Recovery:

Recovery in this ELISA was determined by spiking low and high levels of recombinant human IFNγ into normal human serum, plasma and urine samples as well as a control buffer. Mean recoveries are as follows:

| Spike Level | 900 pg/ml | 150 pg/ml |
|---|---|---|
| Mean Serum Recovery | 106% | 84% |
| Mean Plasma Recovery | 95% | 65% |
| Mean Urine Recovery | 105% | 80% |

8. Expected Values:

Serum, plasma and urine samples were collected from apparently healthy individuals and run in this assay. The levels of human IFNγ detected are reported below.

| | Average | Range |
|---|---|---|
| Serum (n = 35) | 0.3 pg/ml | 0-1.5 pg/ml |
| Plasma (n = 45) | 0.3 pg/ml | 0-2.6 pg/ml |
| Urine (n = 5) | 0.8 pg/ml | 0.5-1.2 pg/ml |

9. STABILITY AND STORAGE

a) Refer to the expiration date stamped on the kit box.

b) Store all reagents at 2.8C upon receiving. Do not freeze reagents.

c) Do not use beyond the stated expiration date.

### ADDENDUM 17

### ANTIOXIDANT CAPACITY ASSAY

Cut the dialysis tubings (approximately 8″), soak in tap water by changing water four to five in 30 minutes. Rinse with distilled water and heat in 5 mM EDTA solution (1.25 mls of 3 g/20 ml solution in 100 ml distilled water) for 3 minutes at 60-70 degrees Centigrade. Rinse with distilled water 2-3 times.

PREPARATION OF METMYGLOBIN

I. Prepare a solution of Potassium ferricyanide, K₃Fe(CN)₆ 740 μM, (2.4 mg/10 ml PBS buffer ph 7.4)

II. Prepare a solution of myoglobin 400 μM, 7.5 mg/ml in 740 μM solution of K₃Fe(CN)₆

Mix and let stand for 5 minutes at room temperature. Transfer this solution by pipette into dialysis bag and dialyze against 400 mls of PBS ph 7.4, for 30 minutes and change of buffer 30 minutes, again after 15 minutes.

Wash the dialysis tube with distilled water. Cut one end and decant off the solution to a sterile 15 ml falcon tube.

CALCULATION OF METMYOGLOBIN CONCENTRATION by WHITBURN'S EQUATION

Dilute 0.1 ml of metmyoglobin solution with 0.9 ml of PBS buffer, in 1 ml plastic cuvette. Invert and mix and read the absorbance at the following wavelengths.

490, 560, 580 and 700 nms against 1 ml PBS ph 7.4 as a blank.

Calculate the concentration of Metmyoglobin by using the following equation.

[Met Mb]=(146A₁₉₀)−(180A₅₆₀)+(2.1A₅₈₀)−(A₇₀₀)

Subtract the absorbance at 700 nm (background). The answer will give you the concentration in a 0.1 ml of Met Mb solution. Multiplying by 10 will give μmoles/L.

### PROTOCOL FOR ANTIOXIDANT CAPACITY

TROLOX solⁿ [2.5 mmoles/L]

| | |
|---|---|
| Stock: | 6.2 mg Trolox in 10 ml PBS pH 7.4 |
| | Sonicate 15-30 min. (keep on ice + dark). |
| Working: | Dilute 100 μl of stock solution + 900 μl of PBS, |
| Fresh (each 3 weeks) | pH 7.4. Daily. |

MetMb solⁿ

| | |
|---|---|
| Working: | Dilute the stock solⁿ to give 70 μmoles/L |
| (Fresh 1-2 wks) | final concentration (ambient temperatures) + dark. |

ABTS:

| | |
|---|---|
| Stock: | (5 mmoles/L) 3 Tablets in 11 ml of PBS, ph 7.4 |
| (Fresh Weekly) | in amber color bottle. (keep on ice) |
| Working: | 2 ml of stock + 8 ml of PBS, pH 7.4 |
| (Fresh Daily) | (ambient temperature, 500 μmoles/L) |

H₂O₂

| | |
|---|---|
| Stock: | (0.098 mole/L) 100 μl of 30% sol + 9.9 mls |
| (Fresh Daily) | of PBS, pH 7.4 (keep on ice) |
| Working: | 50 μl of stock + 10.950 ml of PBS, pH 7.4 |
| (Fresh Daily) | (ambient Temperature, 450 μmoles/L) |

### STEPS

1. Keep plasma on ice, vortex if before addition.

2. Allow PBS, ph 7.4 to reach room temperature.

3. Add 1 ml of PBS, ph 7.4 to a polystyrene cuvette.

4. Turn on spectrophotometer.

5. Set wavelength to 734 nm.

6. Zero the instrument with 1 ml of PBS, ph 7.4

7. Set timer for 6 minutes

8. Cut the parafilm.

ANTI-OXIDANT ASSAY TABLE

| | | Standards | | | | | |
|---|---|---|---|---|---|---|---|
| Reagents | Blank | I | II | III | IV | V | Unknown |
| ABTS (uL) | 300 | 300 | 300 | 300 | 300 | 300 | 300 |
| TROLOX | 0 | 10 | 20 | 30 | 40 | 50 | 0 |
| STANDARD | | | | | | | |
| WORKING | | | | | | | |
| SOLUTION | | | | | | | |
| PLASMA | 0 | 0 | 0 | 0 | 0 | 0 | 0 |
| WORKING | | | | | | | |
| SOLUTION | | | | | | | |
| Met Mb | 36 | 36 | 36 | 36 | 36 | 36 | 36 |
| WORKING | | | | | | | |
| SOLUTION | | | | | | | |
| PBS BUFFER | 497 | 487 | 477 | 467 | 457 | 447 | 487 |
| pH 7.4 | | | | | | | |
| H₂O₂ | 167 | 167 | 167 | 167 | 167 | 167 | 167 |
| WORKING | | | | | | | |
| SOLUTION | | | | | | | |

Timer On
Invert Cuvettes by holding with parafilm
Wait for six (6) minutes
Read immediately at 734 nm

### ADDENDUM 18

Glutathione level test:

Determination of glutathione levels for plasma and/or red blood cells is the preferred test. The test is performed according to Tietze, 1968 Enzymic Method for the Quantitative Determination of Nanogram Amounts of Total and Oxidized Glutathione Analytical Biochemistry with an additional reference of Tietze, 2^{nd} ed., Chemical Chemistry 1994, pp. 1779-1780. This Tietze method has been modified as follows:

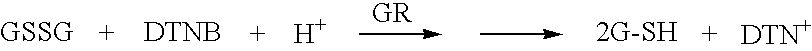

GS⁻+ (2)

where GSSG is gluthathione, oxidized

GR is glutathione reductase

DTNB is a sulfhydryl reagent 5, 5′-dithiobis-(2-nitrobenzoic acid)

G-SH is glutathione, reduced

DTN⁺ is dithiobisnitrobenzoic acid

GS⁻ is a transition state between glutathione reduced and oxidized

The method of glutathione assay provides a sensitive method for total and oxidized glutathione. The modification increases sensitivity for spectrophotometric analysis. The reagents in use throughout this invention, including for this test, are either generally available from a chemical supply house or available from Sigma Chemical Co., Inc. or a company associated with it, Aldrich Chemical Company, of St. Louis, Mo., incorporating DTNB, a sulfhydryl reagent 5, 5′-dithiobis-(2-nitrobenzoic acid) in the first reaction which possesses a molar absorption at 412 mμ then forms two moles of GSH per mole of reduced nucleotide utilized in the GSSG reduction in reaction (2). The rate of chromophore development depends on the concentration of glutathione in the reaction mixture detectable to 10 nanograms ml⁻¹. This provides a highly sensitive and specific procedure for measuring glutathione. The normal level should be approximately 200-400 micromoles/liter for plasma and red blood cells. The test may be performed on an automated clinical chemistry analyser (also called a random access analyzer) such as Roche Cobas Fara. Samples are collected carefully to prevent contamination. Frozen plasma collected from ACD, EDTA, and heparin may be used. The invention could test reduced glutathione but there is not any efficacy over testing total glutathione. Another means of testing glutathione is specifically referenced in Ellerby, L. et al, Measurement of Cellular Oxidation, Reactive Oxygen Species, and Antioxidant Enzymes During Apoptosis, 322 Methods in Enzymology 419-420 (Academic Press 2000).

A discussion of the therapeutic value of appropriate levels in the glutathione pathway is discussed in Rahman I, MacNee W, Free Radical Biological Medicine 2000, May 1, 28(9): 1405-1420.

TABLE II
BASELINE-CBC / Complete Blood Count

| TEST | NORMAL RANGE | | UNITS |
|---|---|---|---|
| WBC | 5-10 | | Thou / CMM |
| RBC | M: 4.6-6.2 | F: 4.2-5.4 | M / μL |
| HgB | M: 14-18 | F: 12-16 | g / dL |
| HCT | M: 40-54 | F: 37-47 | % |

| | | |
|---|---|---|
| MCV | 82-99 | FL |
| MCHC | 33-36 | g / dL |
| RDW | 11.5-14.5 | |
| PLT | 150-400 | k / μL |
| MPV | 6.2-10.8 | FL |
| Lymph | 25-40 | % |
| Mono | 1-8 | % |
| Baso | 0.5-1.0 | % |
| Eosin | 1-4 | % |
| Segs | 50-70 | % |

TABLE III
BASELINE-SMAC / Metabolic Comprehensive Profile

| TEST | NORMAL RANGE | UNITS |
|---|---|---|
| Alk. Phos | 30-103 | U / L |
| Bun | 7-19 | mg / dL |
| Creatinine | 0.7-1.4 | mg / dL |
| Bun / Creatinine ratio | | |
| Glucose | 64-112 | mg / dL |
| Total Protein | 6.0-8.2 | g / dL |
| Uric Acid | 2.1-6.1 | mg / dL |
| Albumin | 3.8-5.2 | g / dL |
| Albumin / Globulin Ratio | | |
| Calcium | 8.5-10.1 | mg / dL |
| Phosphorus | 2.4-4.2 | mg / dL |
| Sodium | 136-145 | mmol / L |
| Potassium | 3.5-5.1 | mmol / L |
| Chloride | 95-106 | mmol / L |
| Bilirubin | 0.0-1.0 | mg / dL |
| LDH | 100-220 | U / L |
| SGOT | 12-29 | U / L |
| SGPT | 9-41 | U / L |
| GGT | 11-55 | U / L |

TABLE IV
Baseline-Lipid/Cardiac Risk

| TEST | NORMAL RANGE | UNITS |
|---|---|---|
| Cholesterol | 140-200 | mg/dL |
| Triglycerides | 40-160 | mg/dL |
| HDL - Cholesterol | see chart | mg/dL |
| LDL - Cholesterol (Calc) | see chart | mg/dL |
| Ferritin | see chart | ng/dL |
| Apolipoprotein A1 | M: 115-190 F: 115-220 | mg/dL |
| (APO A1) | | |
| Apolipoprotein B | M: 70-160 F: 60-150 | mg/dL |
| (APO B) | | |
| APO B/APO A1 Ratio | <1.0 | |
| Lipoprotein-a | 15-30 | mg/dL |
| Homocysteine | 4.0-15.0 | μmole/L |
| Fibrinogen | 200-400 | mg/dL |
| CPK | 41-186 | u/L |

TABLE VIIIA

| HDL CHOLESTEROL mg/dL | | | LDL CHOLESTEROL mg/dL | | |
|---|---|---|---|---|---|
| AGE (YRS) | MALE | FEMALE | AGE (YRS) | MALES | FEMALES |
| 0-14 | 30-65 | 30-65 | 0-19 | 60-140 | 60-150 |
| 15-19 | 30-65 | 30-70 | 20-29 | 60-175 | 60-160 |
| 20-29 | 30-70 | 30-75 | 30-39 | 80-190 | 70-170 |
| 30-39 | 30-70 | 30-80 | 40-49 | 90-205 | 80-190 |
| over 40 | 30-70 | 30-85 | 50-59 | 90-205 | 90-220 |
| | | | 60-69 | 90-215 | 100-235 |
| | | | over 70 | 90-190 | 95-215 |

Values for African-Americans about 10 mg/dL higher

TABLE VIIIB
FERRITIN

| | | | | |
|---|---|---|---|---|
| MALES | 18-45 years | 22-340 ng/dL | >45 years | 22-415 ng/dL |
| FEMALES | 18-45 years | 6-115 ng/dL | >45 years | 15-200 ng/dL |

We claim:

1. An anti-cancer composition for at least one solid tumor cancerous tissue in a patient mammal comprising:
a first combination of white blood cells harvested from a proposed patient's blood sample which has been stabilized, a suitable growth medium, and said at least one cancerous tissue, said first combination then being stimulated with a mitogen challenging compound, and subsequently centrifuged and washed of said mitogen challenging compound to yield a first combination harvest of mitogenically challenged white blood cells;
a second combination composed of a fresh suitable growth medium combined with said first combination harvest, which second combination is heated at a temperature above said patient's normal body temperature at a level to trigger creation of heat shock protein, and then incubated, said second combination thereby yielding upon centrifugation a second combination harvest having a supernatant useable for non-vascular injection which contains immune system markers which biochemically signal said proposed patient's white blood cells to reject and biochemically inhibit growth of said cancerous tissue.

2. The anti-cancer composition according to claim 1, further comprising:
said mitogen-challenging compound being a plant lectin.

3. The anti-cancer composition according to claim 2, further comprising:
said mitogen-challenging compound being PHA.

4. The anti-cancer composition according to claim 3, further comprising:
adding cystine to the supernatant in order to augment immune system response.

5. A method of treating at least one solid tumor cancerous tissue in a patient mammal comprising the following steps:
administering a supernatant by non-vascular injection which contains immune system markers which biochemically signal said proposed patient's white blood cells to reject and biochemically inhibit growth of said cancerous tissue which supernatant has been created from
a) a first combination of a white blood cells harvested from a proposed patient's blood sample which has been stabilized, a suitable growth medium, and said at least one cancerous tissue, said first combination then being stimulated with a mitogen challenging compound, and subsequently centrifuged and washed of said mitogen challenging compound to yield a first combination harvest of mitogenically challenged white blood cells; and
b) a second combination composed of a fresh suitable growth medium combined with said first combination harvest, which second combination is heated at a temperature above said patient's normal body temperature at a level to trigger creation of heat shock protein, and then incubated, said second combination upon centrifugation thereby yielding a second combination harvest having said supernatant.

6. The method according to claim 5, further comprising:
said mitogen-challenging compound being a plant lectin.

7. The method according to claim 6, further comprising:
said mitogen-challenging compound being PHA.

8. The method according to claim 7, further comprising:
adding cystine to the supernatant in order to augment immune system response.

9. A method of treating at least one solid tumor cancerous tissue in a patient mammal comprising the following steps:
harvesting white blood cells for said proposed patient from a blood sample from said patient and testing said white blood cells to determine pre-incubation immune system marker levels;
manufacturing a supernatant for administration by non-vascular injection which contains immune system markers which biochemically signal said proposed patient's white blood cells to reject and biochemically inhibit growth of said cancerous tissue which supernatant has been created from
a) a first combination of a white blood cells harvested from a proposed patient's blood sample which has been stabilized, a suitable growth medium, and said at least one cancerous tissue, said first combination then being stimulated with a mitogen challenging compound, and subsequently centrifuged and washed of said mitogen challenging compound to yield a first combination harvest of mitogenically challenged white blood cells; and
b) a second combination composed of a fresh suitable growth medium combined with said first combination harvest, which second combination is heated at a temperature above said patient's normal body temperature at a level to trigger creation of heat shock protein, and then incubated, said second combination upon centrifugation thereby yielding a second combination harvest having said supernatant;
testing, upon completion of incubation and centrifugation, said supernatant for post-incubation immune system marker levels and comparing said post-incubation immune system marker levels with said pre-incubation immune system marker levels to determine the probable efficacy of said supernatant in treating said at least one solid tumor cancer; and
upon favorable mediation of immune system marker levels as between said pre-incubation levels and said post-incubation levels, periodically administering said supernatant by non-vascular injection.

10. The method according to claim 9, further comprising:
said immune system markers including IL-2 and IL-8.

11. The method according to claim 10, further comprising:
said mitogen-challenging compound being a plant lectin.

12. The method according to claim 11, further comprising:
said mitogen-challenging compound being PHA.

13. The method according to claim 12, further comprising:
adding cystine to the method of treatment in order to augment immune system response.

14. The method according to claim 13, enabling evaluation of variables affecting immune system response to proposed anti-cancer method of treatment and evaluation of efficacy of method of treatment, further comprising the following step:
testing a patient for at least one of a glutathione-S-transferase defect, glutathione level, anti-oxidant capacity, and Karnofsky status.

15. The method according to claim 14, for further determining biochemical pathways needing further mediation to enhance cancer method of treatment:
testing a patient for COX-2 expression, and upon lack of mediation of COX-2 expression, administering a selective COX-2 inhibitor.

16. The method according to claim 15, for further determining biochemical pathways needing further mediation to enhance cancer method of treatment:
testing isoprostane levels in said patient.

17. A method of treating at least one solid tumor cancerous tissue in a patient mammal comprising the following steps:
harvesting white blood cells for said proposed patient from a blood sample from said patient and testing said white blood cells to determine pre-incubation cytokine levels of at least cytokines IL-2 and IL-8;
manufacturing a sup ematant for administration by non-vascular injection which contains immune system markers which biochemically signal said proposed patient's white blood cells to reject and biochemically inhibit growth of said cancerous tissue which supernatant has been created from
a) a first combination of a white blood cells harvested from a proposed patient's blood sample which has been stabilized, a suitable growth medium, a mitogen challenging compound, and said at least one cancerous tissue, said first combination then being stimulated with a mitogen challenging compound, and subsequently centrifuged and washed of said mitogen challenging compound to yield a first combination harvest of mitogenically challenged white blood cells; and
b) a second combination composed of a fresh suitable growth medium combined with said first combination harvest, which second combination is heated at a temperature above said patient's normal body temperature at a level to trigger creation of heat shock protein, and then incubated, said second combination upon centrifugation thereby yielding a second combination harvest having said supernatant;
testing, upon completion of incubation and centrifugation, said supernatant for post-incubation cytokine levels and comparing said post-incubation cytokine levels with said pre-incubation cytokine levels to determine the probable efficacy of said supernatant in modulating immune system reponse to said at least one solid tumor cancer; and
upon favorable mediation of cytokine levels as between said pre-incubation levels and said post-incubation levels, periodically administering said supernatant by non-vascular injection.

18. The method according to claim 17, further comprising:
said mitogen-challenging compound being a plant lectin.

19. The method according to claim 18, further comprising:
said mitogen-challenging compound being PHA.

20. The method according to claim 19, further comprising:
adding cystine to the method of treatment in order to augment immune system response.

21. The method according to claim 20, enabling evaluation of variables affecting immune system response to proposed anti-cancer method of treatment and evaluation of efficacy of method of treatment, further comprising the following step:
testing a patient for at least one of a glutathione-S-transferase defect, glutathione level, anti-oxidant capacity, and Karnofsky status.

22. The method according to claim 21, for further determining biochemical pathways needing further mediation to enhance cancer method of treatment:
testing a patient for COX-2 expression, and upon lack of mediation of COX-2 expression, administering a selective COX-2 inhibitor.

23. The method according to claim 22, for further determining biochemical pathways needing further mediation to enhance cancer method of treatment:
testing isoprostane levels in said patient.

24. An anti-cancer composition for at least one solid tumor cancerous tissue in an immuno-compromised patient mammal comprising:
a first combination of white blood cells harvested from a white blood cell fraction from a blood sample of a non-immuno-compromised mammal of compatible mammalian species and blood type to said patient which sample has been stabilized, a suitable growth medium, and said at least one cancerous tissue, said first combination then being stimulated with a mitogen challenging compound, and subsequently centrifuged and washed of said mitogen challenging compound to yield a first combination harvest of mitogenically challenged white blood cells;
a second combination composed of a fresh suitable growth medium combined with said first combination harvest, which second combination is heated at a temperature above said patient's normal body temperature at a level to trigger creation of heat shock protein, and then incubated, said second combination thereby yielding upon centrifugation a second combination harvest having a supernatant useable for non-vascular injection which contains immune system markers which biochemically signal said proposed patient's white blood cells to reject and biochemically inhibit growth of said cancerous tissue.

25. A method of treating at least one solid tumor cancerous tissue in an immuno-compromised patient mammal comprising the following steps:
separating a white blood cell fraction from a blood sample of a non-immuno-compromised mammal of compatible mammalian species and blood type to said patient;
administering a supernatant to said patient by non-vascular injection which contains immune system markers which biochemically signal said proposed patient's white blood cells to reject and biochemically inhibit growth of said cancerous tissue which supernatant has been created from
a) a first combination of said white blood cell fraction harvested from said non-immuno-compromised mammal which has been stabilized, a suitable growth medium, a mitogen challenging compound, and said at least one cancerous tissue, said first combination then being incubated and subsequently centrifuged and washed of said mitogen challenging compound to yield a first combination harvest of mitogenically challenged white blood cells; and
b) a second combination composed of a fresh suitable growth medium combined with said first combination harvest, which second combination is heated at a temperature above said patient's normal body temperature at a level to trigger creation of heat shock protein, and then incubated, said second combination upon centrifugation thereby yielding a second combination harvest having said supernatant.

26. The method according to claim 25, further comprising:
said immune system markers including IL-2 and IL-8.

27. The method according to claim 26, further comprising:
said mitogen-challenging compound being a plant lectin.

28. The method according to claim 27, further comprising:
said mitogen-challenging compound being PHA.

29. The method according to claim 28, further comprising:
adding cystine to the method of treatment in order to augment immune system response.

30. A method of treating at least one solid tumor cancerous tissue in an immuno-compromised patient mammal comprising the following steps:
separating a white blood cell fraction from a blood sample of a non-immuno-compromised mammal of compatible mammalian species and blood type to said patient;
harvesting white blood cells for said proposed patient from a blood sample from said patient and testing said white blood cells to determine pre-incubation immune system marker levels;
manufacturing a supernatant for administration by non-vascular injection which contains immune system markers which biochemically signal said proposed patient's white blood cells to reject and biochemically inhibit growth of said cancerous tissue which supernatant has been created from
a) a first combination of said white blood cell fraction harvested from said non-immuno-compromised mammal which has been stabilized, a suitable growth medium, and said at least one cancerous tissue, said first combination then being stimulated with a mitogen challenging compound, and subsequently centrifuged and washed of said mitogen challenging compound to yield a first combination harvest of mitogenically challenged white blood cells; and
b) a second combination composed of a fresh suitable growth medium combined with said first combination harvest, which second combination is heated at a temperature above said patient's normal body patient at a level to trigger creation of heat shock protein, and then incubated, said second combination upon centrifugation thereby yielding a second combination harvest having said supernatant;
testing, upon completion of incubation and centrifugation, said supernatant for post-incubation immune system marker levels and comparing said post-incubation immune system marker levels with said pre-incubation immune system marker levels to determine the probable efficacy of said supernatant in treating said at least one solid tumor cancer; and
and upon favorable mediation of immune system marker levels as between said pre-incubation levels and said post-incubation levels, periodically administering said supernatant by non-vascular injection.

31. The method according to claim 29, further comprising:
said immune system markers including IL-2 and IL-8.

32. The method according to claim 31, further comprising:
said mitogen-challenging compound being a plant lectin.

33. The method according to claim 32, further comprising:
said mitogen-challenging compound being PHA.

34. The method according to claim 33, further comprising:
adding cystine to the method of treatment in order to augment immune system response.

35. The method according to claim 34, enabling evaluation of variables affecting immune system response to proposed anti-cancer method of treatment and evaluation of efficacy of method of treatment, further comprising the following step:
testing a patient for at least one of a glutathione-S-transferase defect, glutathione level, anti-oxidant capacity, and Kamofsky status.

36. The method according to claim 35, for further determining biochemical pathways needing further mediation to enhance cancer method of treatment:
testing a patient for COX-2 expression, and upon lack of mediation of COX-2 expression, administering a selective COX-2 inhibitor.

37. The method according to claim 36, for further determining biochemical pathways needing further mediation to enhance cancer method of treatment:
testing isoprostane levels in said patient.

38. A method of treating at least one solid tumor cancerous tissue in an immuno-compromised patient mammal comprising the following steps:
separating a white blood cell fraction from a blood sample of a non-immuno-compromised mammal of compatible mammalian species and blood type to said patient;
harvesting white blood cells for said proposed patient from a blood sample from said patient and testing said white blood cells to determine pre-incubation cytokine levels of at least cytokines IL-2 and IL-8;
manufacturing a supernatant for administration by non-vascular injection which contains immune system markers which biochemically signal said proposed patient's white blood cells to reject and biochemically inhibit growth of said cancerous tissue which supernatant has been created from
a) a first combination of said white blood cell fraction harvested from said non-immuno-compromised mammal which has been stabilized, a suitable growth medium,and said at least one cancerous tissue, said first combination then being stimulated with a a mitogen challenging compound, and subsequently centrifuged and washed of said mitogen challenging compound to yield a first combination harvest of mitogenically challenged white blood cells; and
b) a second combination composed of a fresh suitable growth medium combined with said first combination harvest, which second combination is heated at a temperature above said patient's normal body patient at a level to trigger creation of heat shock protein, and then incubated, said second combination upon centrifugation thereby yielding a second combination harvest having said supernatant;
testing, upon completion of incubation and centrifugation, said supernatant for post-incubation cytokine levels and comparing said post-incubation cytokine levels with said pre-incubation cytokine levels to determine the probable efficacy of said supernatant in treating said at least one solid tumor cancer; and
and upon favorable mediation of levels of cytokines as between said pre-incubation levels and said post-incubation levels, periodically administering said supernatant by non-vascular injection.

39. The method according to claim 38, further comprising:
said mitogen-challenging compound being a plant lectin.

40. The method according to claim 39, further comprising:
said mitogen-challenging compound being PHA.

41. The method according to claim 40, further comprising:
adding cystine to the method of treatment in order to augment immune system response.

42. The method according to claim 41, enabling evaluation of variables affecting immune system response to proposed anti-cancer method of treatment and evaluation of efficacy of method of treatment, further comprising the following step:
testing a patient for at least one of a glutathione-S-transferase defect, glutathione level, anti-oxidant capacity, and Karnofsky status.

43. The method according to claim 42, for further determining biochemical pathways needing further mediation to enhance cancer method of treatment:
testing a patient for COX-2 expression, and upon lack of mediation of COX-2 expression, administering a selective COX-2 inhibitor.

44. The method according to claim 43, for further determining biochemical pathways needing further mediation to enhance cancer method of treatment:
testing isoprostane levels in said patient.

45. A composition for creating a comparable first and second fraction for in vitro testing of efficacy of an anti-cancer therapy in a mammal against at least one particular cancer line, said fractions containing immune system markers, said composition comprising:
a first combination of white blood cells and ambient immune system markers harvested from a blood sample for which first combination a set of immune system marker levels is determined, including at least IL-2 and IL-8;
a second combination of a fresh growth medium, a portion of said cancerous tissue sample, said first combination, and said vaccine in a suitable growth medium, which is incubated, yielding upon centrifugation a second combination harvest having a supernatant;
said supernatant being capable of being compared for levels of immune system markers with levels of immune system markers from said first combination.

46. A method of ex vivo rapid testing of efficacy of an anti-cancer therapy in a mammalian patient against at least one particular cancer line, comprising the following steps:
prior to initiation of said therapy, harvesting a blood sample from said patient and separating white blood cells from red blood cells to generate pre-therapy white blood cells;
determining for said pre-therapy white blood cells a selected set of immune system marker levels including at least IL-2 and 11-8;
subsequent to initiation of therapy according to the proposed therapy regimen, harvesting at least one new sample of said patient's blood and determining for said at least one subsequent sample of white blood cells said selected set of immune system marker levels;
gauging successful mediation of immune system marker levels by comparing said selected set of immune system marker levels in said pre-therapy white blood cells with immune system marker levels of at least one of said at least one subsequent sample.

47. The method according to claim 40, enabling evaluation of variables affecting immune system response to proposed anti-cancer method of treatment and evaluation of efficacy of method of treatment, further comprising the following step:
testing a patient for at least one of a glutathione-S-transferase defect, glutathione level, anti-oxidant capacity, and Karnofsky status.

48. The method according to claim 41, for further determining biochemical pathways needing further mediation to enhance cancer method of treatment:
testing a patient for COX-2 expression, and upon lack of mediation of COX-2 expression, recommending a selective COX-2 inhibitor.

49. A method of manufacturing a cytokine modulator for treating at least one solid tumor cancerous tissue in a patient mammal comprising the following steps:
harvesting white blood cells for said proposed patient from a blood sample from said patient;
combining white blood cells harvested from a proposed patient's blood sample which has been stabilized, a suitable growth medium, a mitogen challenging compound, and said at least one cancerous tissue, incubating and subsequently centrifuging, and then washing out said mitogen challenging compound to yield a first combination harvest of mitogenically challenged white blood cells;
combining a fresh suitable growth medium with said first combination harvest to generate a second combination, which second combination is heated at a temperature above said patient's normal body temperature at a level to trigger creation of heat shock protein, incubating such second combination and separating off by centrifugation supernatant generated from such incubation.

50. The method according to claim 49, further comprising:
said mitogen-challenging compound being a plant lectin.

51. The method according to claim 50, further comprising:
said mitogen-challenging compound being PHA.

52. The method according to claim 52, further comprising:
adding cystine to the method of manufacture in order to augment immune system response.

53. A method of manufacturing a cytokine modulator for treating at least one solid tumor cancerous tissue in a patient mammal comprising the following steps:
harvesting white blood cells for said proposed patient from a blood sample from said patient and testing said white blood cells to determine pre-incubation immune system marker levels;
combining white blood cells harvested from a proposed patient's blood sample which has been stabilized, a suitable growth medium, a mitogen challenging compound, and said at least one cancerous tissue, incubating and subsequently centrifuging, and then washing out said mitogen challenging compound to yield a first combination harvest of mitogenically challenged white blood cells;
combining a fresh suitable growth medium with said first combination harvest to generate a second combination, which second combination is heated at a temperature above said patient's normal body temperature at a level to trigger creation of heat shock protein, incubating such second combination and separating off by centrifugation supernatant generated from such incubation;
testing, upon completion of incubation and centrifugation, said supernatant for post-incubation immune system marker levels and comparing said post-incubation immune system marker levels with said pre-incubation immune system marker levels to determine the probable efficacy of said supernatant in treating said at least one solid tumor cancer.

54. The method according to claim 53, further comprising:
said immune system markers including IL-2 and IL-8.

55. The method according to claim 54, further comprising:
said mitogen-challenging compound being a plant lectin.

56. The method according to claim 55, further comprising:
said mitogen-challenging compound being PHA.

57. The method according to claim 56, further comprising:
adding cystine to the method of manufacture in order to augment immune system response.

58. A method of manufacturing a cytokine modulator for treating at least one solid tumor cancerous tissue in a patient mammal comprising the following steps:
harvesting white blood cells for said proposed patient from a blood sample from said patient and testing said white blood cells to determine pre-incubation cytokine levels of at least cytokines IL-2 and IL-8;
combining white blood cells harvested from a proposed patient's blood sample which has been stabilized, a suitable growth medium, and said at least one cancerous tissue, stimulating with a mitogen challenging compound, and subsequently centrifuging, and then washing out said mitogen challenging compound to yield a first combination harvest of mitogenically challenged white blood cells;
combining a fresh suitable growth medium with said first combination harvest to generate a second combination, which second combination is heated at a temperature above said patient's normal body temperature at a level to trigger creation of heat shock protein, incubating such second combination and separating off by centrifugation supernatant generated from such incubation;
testing, upon completion of incubation and centrifugation, said supernatant for post-incubation cytokine levels and comparing said post-incubation cytokine levels with said pre-incubation cytokine levels to determine the probable efficacy of said supernatant in treating said at least one solid tumor cancer.

59. The method according to claim 58, further comprising:
said mitogen-challenging compound being a plant lectin.

60. The method according to claim 59, further comprising:
said mitogen-challenging compound being PHA.

61. The method according to claim 60, further comprising:
adding cystine to the method of manufacture in order to augment immune system response.

62. A method of manufacturing a cytokine modulator for treating at least one solid tumor cancerous tissue in a patient mammal comprising the following steps:
harvesting white blood cells for said proposed patient from a blood sample from said patient and testing said white blood cells to determine pre-incubation cytokine levels of at least cytokines IL-2 and IL-8;
combining white blood cells harvested from a proposed patient's blood sample which has been stabilized, a suitable growth medium, and said at least one cancerous tissue, stimulating with a plant lectin, and subsequently centrifuging, and then washing out said plant lectin to yield a first combination harvest of mitogenically challenged white blood cells;
combining a fresh suitable growth medium with said first combination harvest to generate a second combination, which second combination is heated at a temperature above said patient's normal body temperature at a level to trigger creation of heat shock protein, incubating such second combination and separating off by centrifugation supernatant generated from such incubation;
testing, upon completion of incubation and centrifugation, said supernatant for post-incubation cytokine levels and comparing said post-incubation cytokine levels with said pre-incubation cytokine levels to determine the probable efficacy of said supernatant in treating said at least one solid tumor cancer.

63. A method of manufacturing a cytokine modulator for treating at least one solid tumor cancerous tissue in a patient mammal comprising the following steps:
harvesting white blood cells for said proposed patient from a blood sample from said patient into a tube having blood stabilizer and a thixotropic medium to facilitate discreet harvesting of white blood cells after centrifugation because of improved separation by density medium centrifugation into distinct cell populations;
testing said white blood cells to determine pre-incubation cytokine levels of at least cytokines IL-2, IL-8, and IL-10;
combining white blood cells harvested from said proposed patient's blood sample which has been stabilized, a suitable growth medium, and said at least one cancerous tissue, stimulating with PHA for one hour, and subsequently centrifuging, and then washing out said PHA with NaCl solution three times to yield a first combination harvest of mitogenically challenged white blood cells;
combining a fresh suitable growth medium with said first combination harvest to generate a second combination, which second combination is heated at a temperature for one hour above said patient's normal body temperature at a level to trigger creation of heat shock protein, incubating such second combination for at least 96 hours and separating off by centrifugation supernatant generated from such incubation;
testing, upon completion of incubation and centrifugation, said supernatant for post-incubation cytokine levels and comparing said post-incubation cytokine levels with said pre-incubation cytokine levels to determine the probable efficacy of said supernatant in treating said at least one solid tumor cancer.

64. A method of manufacturing a cytokine modulator for treating at least one solid tumor cancerous tissue in an immuno-compromised patient mammal comprising the following steps:
separating a white blood cell fraction from a blood sample of a non-immuno-compromised mammal of compatible mammalian species and blood type to said patient;
testing said white blood cell fraction to determine pre-incubation immune system marker levels;
combining white blood cells from said white blood cell fraction which has been stabilized, a suitable growth medium, and said at least one cancerous tissue, stimulating with a mitogen challenging compound, and subsequently centrifuging, and then washing out said mitogen challenging compound to yield a first combination harvest of mitogenically challenged white blood cells;
combining a fresh suitable growth medium with said first combination harvest to generate a second combination, which second combination is heated at a temperature above said patient's normal body temperature at a level to trigger creation of heat shock protein, incubating such second combination and separating off by centrifugation supernatant generated from such incubation; testing, upon completion of incubation and centrifugation, said supernatant for post-incubation immune system marker levels and comparing said post-incubation immune system marker levels with said pre-incubation immune system marker levels to determine the probable efficacy of said supernatant in treating said at least one solid tumor cancer.

65. A method of manufacturing a cytokine modulator for treating at least one solid tumor cancerous tissue in an immuno-compromised patient mammal comprising the following steps:
separating a white blood cell fraction from a blood sample of a non-immuno-compromised mammal of compatible mammalian species and blood type to said patient;
testing said white blood cell fraction to determine pre-incubation cytokine levels of at least cytokines IL-2 and IL-8;
combining white blood cells from said white blood cell fraction which has been stabilized, a suitable growth medium, and said at least one cancerous tissue, stimulating with a mitogen challenging compound and subsequently centrifuging, and then washing out said mitogen challenging compound to yield a first combination harvest of mitogenically challenged white blood cells;
combining a fresh suitable growth medium with said first combination harvest to generate a second combination, which second combination is heated at a temperature above said patient's normal body temperature at a level to trigger creation of heat shock protein, incubating such second combination and separating off by centrifugation supernatant generated from such incubation;
testing, upon completion of incubation and centrifugation, said supernatant for post-incubation cytokine levels and comparing said post-incubation cytokine levels with said pre-incubation cytokine levels to determine the probable efficacy of said supernatant in treating said at least one solid tumor cancer.

66. The method according to claim 54, further comprising:
said mitogen-challenging compound being a plant lectin.

67. The method according to claim 55, further comprising:
said mitogen-challenging compound being PHA.

68. The method according to claim 56, further comprising:
adding cystine to the method of manufacture in order to augment immune system response.

69. The composition according to claims 1, 2, 3, 4, 24, and 45, further comprising:
said blood sample being combined in a tube with a thixotropic medium to enable, upon centrifugation, more discreet harvest of said white blood cells harvested for said first combination.

70. The method according to claims 5,6,7,8,9,10,11,12,13,17, 18, 19, 20, 22, 23, 25, 26,27,28,29, 30, 31, 32, 33, 34, 36, 37, 38, 39, 40, 41, 43, 44, 48, 49, 50, 51, 52, 53,54,55,56,57,58, 60, 61, 62, 64, 65, 66, 67, and 68, further comprising:
said step of harvesting white blood cells being a harvesting of a blood sample from the patient into a tube having a thixotropic medium to facilitate discreet harvesting of white blood cells after centrifugation because of improved separation by density medium centrifugation into distinct cell populations and centrifugation.
